(19) United States
(12) Patent Application Publication
(10) Pub. No.: US 2007/0219239 A1
(43) Pub. Date: Sep. 20, 2007

(54) Nitrogen-containing heterocycle derivatives, pharmaceutical compositions, and methods of use thereof as antiviral agents

(76) Inventors: Adnan M.M. Mjalli, Oak Ridge, NC (US); Jeremy T. Cooper, High Point, NC (US); Murty N. Arimilli, Oak Ridge, NC (US); Robert C. Andrews, Jamestown, NC (US); Robert Rothlein, Sumerfield, NC (US); Taleb H. AlTel, Greensboro, NC (US)

Correspondence Address: KILPATRICK STOCKTON LLP - 41305;CHARLES CALKINS, 1001 WEST FOURTH STREET, WINSTON-SALEM, NC, 27101, US

(21) Appl. No.: 11/704,763

(22) Filed: Feb. 9, 2007

Related U.S. Application Data
(60) Provisional application No. 60/772,309, filed on Feb. 10, 2006.

(51) Int. Cl. A61K 31/4709; A61K 31/4439; A61K 31/4164; C07D 403/04

(52) U.S. Cl. 514/307; 514/396; 514/314; 514/341; 546/148; 546/169; 546/273.1; 548/335.5

(57) ABSTRACT

The present application provides nitrogen-containing heterocycle derivatives that are antiviral compounds that may be useful in the treatment of a viral infection. Compounds of Formula (I) and pharmaceutical compositions comprising a compound of Formula (I) may be administered to a subject for antiviral therapy or prophylaxis.

### CROSS-REFERENCE TO RELATED APPLICATIONS

This application claims the benefit of priority under 35 U.S.C. § 119(e) of U.S. Provisional Application No. 60/772,309 filed Feb. 10, 2006, entitled “Nitrogen-Containing Heterocycle Derivatives, Pharmaceutical Compositions, and Methods of Use Thereof as Antiviral Agents”, the disclosure of which is herein incorporated by reference in its entirety.

### STATEMENT REGARDING FEDERALLY SPONSORED RESEARCH

The invention disclosed herein was made with Government support under Grant Number 1 R43 AI060151-01 from the National Institutes of Health, U.S. Department of Health and Human Services. Accordingly, the U.S. Government has certain rights in this invention.

### FIELD OF THE INVENTION

The present invention provides nitrogen containing heterocycle compounds. These compounds are antiviral agents and may be useful in the treatment of a viral infection in a subject or prophylaxis.

### BACKGROUND OF THE INVENTION

Viral replication can be thought of as consisting of two phases. The early phase of viral infection consists of entry into a host cell, the decision between lysogeny and lysis, and then replication itself. Subsequent synthesis and assembly of structural proteins into the finished virions occurs during the late phase of infection. Almost all viruses carefully regulate their gene expression to correctly time the expression of early- and late-phase genes. Interception of steps in process of viral replication may cripple viral propagation.

While useful antiviral agents have been identified, there still exists a need in the art for compounds that demonstrate desirable antiviral activity against one or more viruses.

### BRIEF SUMMARY OF THE INVENTION

Embodiments of the present invention provide nitrogen-containing heterocycle derivatives, compositions comprising the same, and methods of using such compounds and compositions as antiviral agents.

In one aspect, the present invention provides compounds of Formula (I) as shown below. In another aspect, the present invention provides methods for the preparation of compounds of Formula (I).

In another aspect, the present invention provides pharmaceutical compositions comprising a compound of Formula (I). In an embodiment, the pharmaceutical composition comprises a compound of Formula (I) and a pharmaceutically acceptable carrier, excipient, diluent, or a mixture thereof. In another aspect, the present invention provides a method for the preparation of a pharmaceutical composition comprising a compound of Formula (I).

In another aspect, the present invention provides methods for using a compound of Formula (I) or a pharmaceutical composition comprising a compound of Formula (I) as an antiviral agent. In an embodiment, a compound of Formula (I) or a pharmaceutical composition comprising a compound of Formula (I) is administered to a subject in need thereof.

Additional features of the present invention are described hereinafter.

### DETAILED DESCRIPTION OF THE INVENTION

The compounds of the present invention are useful in the treatment or prophylaxis of one or more viral infections in a subject. Viral infections that may be treated by the compounds and pharmaceutical compositions of the present invention include, but are not limited to, a viral infections caused by a DNA virus or an RNA virus.

DNA viruses include, but are not limited to, Adenoviridae including adenovirus, Hepadnaviridae including hepatitis B virus (HBV), Herpesviridae including herpes simplex virus type 1 (HSV-1), type 2 (HSV-2), thymidine kinase-deficient (TK⁻) HSV-1, varicella-zoster virus (TK⁺ and TK⁻ VZV), cytomegalovirus (CMV), human herpesvirus type 6 (HHV-6), and feline herpesvirus, Poxviridae including vaccinia virus, Papillomaviridae including human papilloma virus, and Polyomaviridae including polyoma virus; and

RNA viruses include, but are not limited to, Retroviridae including human immunodeficiency virus type 1 (HIV-1) and type 2 (HIV-2), simian immunodeficiency virus (SIV), and moloney murine sarcoma virus, Coronaviridae including feline (FIPV) corona virus, human (SARS) CoV, and mouse hepatitis virus, Flaviviridae including flavivirus (yellow fever virus (YFV), dengue-type 2 virus, and modoc virus (murine flavivirus)), hepacivirus (hepatitis C, hepatitis A, hepatitis B), and pestivirus (bovine viral diarrhea virus (BVDV)), Picornaviridae including coxsackie B virus, polio virus, and rhinovirus, Alphaviridae including sindbis virus, Arenaviridae including arenaviruses (Tacaribe), Bunyaviridae including punta toro, Orthomyxoviridae including influenza A, B, and C virus, Paramyxoviridae including respiratory syncytial virus (RSV) and parainfluenza-3 virus, and Reoviridae including reo-1 virus.

In a one aspect, the present invention provides a compound of Formula (I): 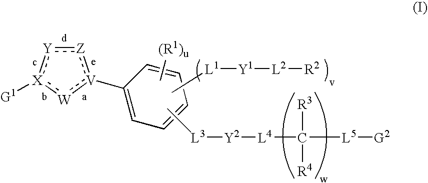
wherein
V is C, W is N—R¹¹, O, or S, X is C, Y is N, Z is C—R¹², when sides a, b, and d are single bonds, and sides c and e are double bonds;
V is C, W is N, X is C, Y is N—R¹¹, O, or S, Z is C—R¹², when sides a, c, and d are single bonds, and sides b and e are double bonds;
V is C, W is N, X is C, Y is C—R¹², Z is N—R¹¹, O, or S when sides b, e, and d are single bonds, and sides a and c are double bonds;
V is C, W is C—R¹², X is N, Y is C—R¹³, Z is N, when sides b, c, and e are single bonds, and sides a and d are double bonds;
V is N, W is C—R¹², X is C, Y is N, Z is C—R¹³, when sides a, c, and e are single bonds, and sides b and d are double bonds;
V is C, W is N—R¹¹, O, or S, X is C, Y is C—R¹², Z is N when sides a, b, and d are single bonds, and sides c and e are double bonds;
   wherein
      R¹¹ is R^{d}
      R¹² and R¹³ are independently selected from R^{f},
G¹ is selected from the group consisting of: cycloalkyl, heterocyclyl, aryl, heteroaryl, fused arylcycloalkyl, fused cycloalkylaryl, fused cycloalkylheteroaryl, fused heterocyclylaryl, and fused heterocyclylheteroaryl group, wherein G¹ is optionally substituted with substituents independently selected from R⁵, wherein R⁵ is R^{b},
G² is selected from the group consisting of: cycloalkyl, heterocyclyl, aryl, heteroaryl, fused arylcycloalkyl, fused cycloalkylaryl, fused cycloalkylheteroaryl, fused heterocyclylaryl, and fused heterocyclylheteroaryl group, wherein G² is optionally substituted with substituents independently selected from R⁶, wherein R⁶ is R^{b},
R¹ is R^{b},
R² is R^{f},
R³ and R⁴ are independently selected from R^{f} and R^{g},
L¹, L², and L⁵ are independently selected from the group consisting of a direct bond, —C₁₋₁₀ alkylene, —C₂₋₁₀ alkenylene, and —C₂₋₁₀ alkynylene; wherein alkylene, alkenylene, and alkynylene are optionally substituted 1 to 4 times with R^{f},
L³ and L⁴ are independently selected from the group consisting of a direct bond, —C₁₋₁₀ alkylene, —C₂₋₁₀ alkenylene, —C₂₋₁₀ alkynylene, arylene, and heteroarylene; wherein alkylene, alkenylene, and alkynylene are optionally substituted 1 to 4 times with R^{f}, and arylene and heteroarylene are optionally substituted 1 to 4 times with R^{b},
Y¹ and Y² are independently selected from the group consisting of a direct bond, —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, —C(R^{f})═C(R^{g})—, —C≡C—, —N═N—, and —N(R¹⁶)—N(R¹⁷)—;
   wherein
   R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are optionally substituted 1 to 4 times with R^{f};
R^{b} is
   a) -cycloalkyl,
   b) -cyano,
   c) —OR^{d},
   d) —NO₂,
   e) -halogen,
   f) —S(O)ₘR^{d},
   g) —SR^{d},
   h) —S(O)₂OR^{d},
   i) —S(O)ₘNR^{d}R^{e},
   j) —NR^{d}R^{e},
   k) —O(CR^{f}R^{g})ₙNR^{d}R^{e},
   l) —C(O)R^{d},
   m) —CO₂R^{d},
   n) —CO₂(CR^{f}R^{g})ₙC(O)NR^{d}R^{e},
   o) —OC(O)R^{d},
   p) —C(O)NR^{d}R^{e},
   q) —NR^{d}C(O)R^{e},
   r) —OC(O)NR^{d}R^{e},
   s) —NR^{d}C(O)OR^{e},
   t) —NR^{d}C(O)NR^{d}R^{e},
   u) —CF₃,
   v) —OCF₃,
   w) -haloalkyl,
   x) -haloalkoxy,
   y) —C₁₋₁₀ alkyl,
   z) —C₂₋₁₀ alkenyl,
   aa) —C₂₋₁₀ alkynyl,
   bb) —C₁₋₁₀ alkylene-aryl,
   cc) —C₁₋₁₀ alkylene-heteroaryl, or
   dd) -heteroaryl,
   wherein alkyl, alkenyl, alkynyl, aryl, heteroaryl, and cycloalkyl groups are optionally substituted 1-4 times with a group independently selected from R^{c};
R^{c} is
   a) -halogen,
   b) -amino,
   c) -carboxy,
   d) —C₁₋₄ alkyl,
   e) —O—C₁₋₄ alkyl,
   f) -cycloalkyl,
   g) —O-cycloalkyl,
   h) -aryl,
   i) —C₁₋₄ alkylene-aryl,
   j) -hydroxy,
   k) —CF₃,
   l) -haloalkyl,
   m) -haloalkoxy,
   n) —O-aryl,
   o) -heteroaryl,
   p) -heteroaryl-C₁₋₁₀ alkyl,
   q) heterocyclyl,
   r) —CO₂—C₁₋₁₀ alkyl, or
   s) —CO₂—C₁₋₁₀ alkyl-aryl,
R^{d} and R^{e} are independently selected from hydrogen, C₁₋₁₀ alkyl, C₂₋₁₀ alkenyl, C₂₋₁₀ alkynyl, cycloalkyl, —C₁₋₁₀ alkylene-cycloalkyl, aryl, heterocyclyl, wherein alkyl, alkenyl, alkynyl, cycloalkyl, aryl, heterocyclyl groups are optionally substituted with one to four substituents independently selected from R^{c}; or R^{d}and R^{e} together with the atoms to which they are attached form a heterocyclic ring of 5 to 7 members containing 0-2 additional heteroatoms independently selected from oxygen, sulfur and nitrogen and optionally substituted with 1-3 times with R^{c},
R^{f} and R^{g} are independently selected from hydrogen, C₁₋₁₀ alkyl, cycloalkyl, —C₁₋₁₀ alkylene-cycloalkyl, and aryl, wherein alkyl, cycloalkyl, and aryl groups are optionally substituted with one to four substituents independently selected from R^{c}; or R^{f} and R^{g} together with the carbon to which they are attached form a ring of 5 to 7 members containing 0-2 heteroatoms independently selected from oxygen, sulfur and nitrogen optionally substituted with 1-3 times with R^{c};
m is an integer from 1 to 2,
n is an integer from 1 to 10,
u is an integer from 0 to 2,
v is an integer from 0 to 2,
w is an integer from 0 to 1,
or pharmaceutically acceptable salt, solvate, or prodrug thereof.
In an embodiment, G¹ is isoquinoline, quinoline, quinazoline, cinnoline, purine, tetrahydroisoquinoline, indole, isoindole, indoline, pyridine, pyrimidine, pyridazine, pyrazine, benzimidazole, benzothiazole, benzoxazole, imidazole, pyrrole, thiazole, oxazole, isothiazole, phenyl, or naphthyl, wherein G¹ is optionally substituted 1 to 4 times with R⁵, wherein R⁵ is R^{b}.
In another embodiment, G¹ is heterocyclyl, heteroaryl, fused cycloalkylheteroaryl, fused heterocyclylaryl, or fused heterocyclylheteroaryl ring containing at least one nitrogen atom, wherein the nitrogen atom in G¹ is ortho to the atom in G¹ connected to the remainder of the compound of Formula (I). In a further embodiment, G¹ is a heterocyclyl, heteroaryl, fused cycloalkylheteroaryl, fused heterocyclylaryl, or fused heterocyclylheteroaryl ring containing at least one nitrogen atom, wherein the nitrogen atom in G¹ is ortho to the atom in G¹ connected to the remainder of the compound of Formula (I) and G¹ is connected to the 2 position of an imidazole ring represented by V, W, X, Y, and Z.
In another embodiment, G¹ is isoquinoline-3-yl, quinoline-2-yl, quinoline-3-yl, pyridine-2-yl, pyridine-3-yl, phenyl, or naphthyl-2-yl, wherein G¹ may be substituted or unsubstituted. In a further embodiment, G¹ is isoquinoline-3-yl, pyridine-2-yl, pyridine-3-yl, or phenyl, wherein G¹ may be substituted or unsubstituted. In an further embodiment, G¹ is isoquinoline-3-yl, pyridine-2-yl, or pyridine-3-yl, wherein G¹ is unsubstituted.
In an embodiment, G² is phenyl, pyridine, pyrimidine, pyridazine, or pyrazine, wherein G² is optionally substituted 1 to 4 times with R⁶, wherein R⁶ is R^{b}.
In an embodiment, V is C, W is N—R¹¹, O, or S, X is C, Y is N, Z is C—R¹², sides a, b, and d are single bonds, and sides c and e are double bonds. In a further embodiment, V is C, W is N—R¹¹, X is C, Y is N, Z is C—R ¹², sides a, b, and d are single bonds, and sides c and e are double bonds. In a further embodiment, R¹¹ and R¹² are independently selected from the group consisting of hydrogen, C₁₋₁₀ alkyl, cycloalkyl, phenyl, and —C₁₋₁₀ alkylene-phenyl. In a further embodiment, R¹¹ and R¹² are hydrogen. p0 In another embodiment, u is 0, and v is 1. In another embodiment, u is 0, and v is 0. In another embodiment, u is 1, and v is 0.
In another embodiment,
   u is 0,
   v is 1,
   L¹ is a direct bond,
   L² is a direct bond,
   Y¹ is —N(R¹⁶)C(O)—, and
   R² is R^{f}.
In another embodiment,
   u is 0,
   v is 1,
   L¹ is a direct bond,
   L² is a direct bond,
   R² is hydrogen, C₁₋₁₀ alkyl, cycloalkyl, phenyl, or —C₁₋₁₀ alkylene-phenyl, wherein alkyl, cycloalkyl, and phenyl are substituted or unsubstituted, and
   Y¹ is —N(R¹⁶)C(O)—, wherein R¹⁶ is hydrogen, C₁₋₁₀ alkyl, cycloalkyl, or phenyl, wherein alkyl, cycloalkyl, and phenyl are substituted or unsubstituted.
In another embodiment,
   u is 0,
   v is 1,
   L¹ is a direct bond,
   L² is a direct bond,
   Y¹ is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, and —C(R^{f})═C(R^{g})—;
      wherein
         R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted; and
   R² is C₁₋₁₀ alkyl, cycloalkyl, phenyl, or —C₁₋₁₀ alkylene-phenyl, wherein alkyl, cycloalkyl, and phenyl are substituted or unsubstituted.
In another embodiment,
   u is 0,
   v is 1,
   L¹ is a direct bond,
   L² is a direct bond,
   Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted;
   R² is C₁₋₁₀ alkyl, -cycloalkyl, -phenyl, or —C₁₋₁₀ alkylene-phenyl, wherein alkyl, cycloalkyl, and phenyl are substituted or unsubstituted.
In another embodiment,
   u is 0,
   v is 1,
   L¹ is a direct bond,
   L² is a direct bond,
   Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted; and
   R² is methyl, ethyl, propyl, isopropyl, tert-butyl, isobutyl, 1-ethyl-propyl, and (1-halo-1-methyl)-ethyl.
In another embodiment, L³, L⁴, and L⁵ are direct bonds, and w is 1.
In another embodiment, L³, L⁴, and L⁵ are direct bonds, w is 1, and Y² is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, and —C(R^{f})═C(R^{g})—;
   wherein
      R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.
In another embodiment, L³, L⁴, and L⁵ are direct bonds, w is 1, and Y² is selected from the group consisting of a —C(O)N(R¹⁶)—, —N(R¹⁶)C(O)—, —N(R¹⁶)SO₂—, —SO₂N(R¹⁶)——C(O)—O—, —O—C(O)—, and —C(R^{f})═C(R^{g})—;
   wherein
      R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.
In another embodiment,
   L³, L⁴, and L⁵ are direct bonds,
   w is 1,
   Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted;
   R³ is H,
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
   G² is phenyl substituted from 1 to 4 times with R⁶.
In another embodiment,
   L³, L⁴, and L⁵ are direct bonds,
   w is 1,
   Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
   R³ is H,
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
   G² is phenyl substituted from 1 to 4 times with R⁶, wherein G² is substituted with at least one halogen.
In another embodiment,
   L³, L⁴, and L⁵ are direct bonds,
   w is 1,
   Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted;
   R³ is H,
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
   G² is para-halophenyl.
In another embodiment, the group -L³-Y²-L⁴-(C(R³)(R⁴))-L⁵-G² is taken together to form the group 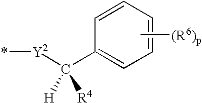
   wherein
   Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
   R⁴ is R^{b},
   R⁶ is R^{b}, and
   p is an integer from 0 to 4.
   In a further embodiment, Y² is —C(O)NH—, R⁴ is —C₁₋₄ alkyl, p is 1, and R⁶ is halo.
In another embodiment, the group -L³-Y²-L⁴-(C(R³)(R⁴))-L⁵-G² is taken together to form the group 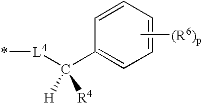
   wherein
   L⁴ is imidazole, oxazole, or thiazole,
   R⁴ is R^{b},
   R⁶ is R^{b}, and
   p is an integer from 0 to 4.
   In a further embodiment, Y² is imidazole, R⁴ is —C₁₋₄ alkyl, p is 1, and R⁶ is halo.
In another embodiment, the compound of Formula (I) has the formula (Ia) 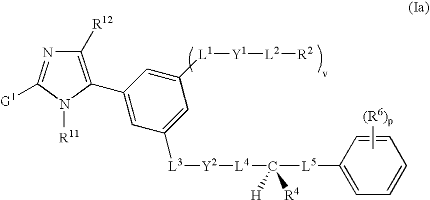
   wherein
   G¹, R², R⁴, R⁵, R¹¹, R¹², L¹, L², L³, L⁴, L⁵, Y¹, Y², and v are as defined above, and
   p is an integer from 0 to 4.
In another embodiment of the compound of Formula (Ia), G¹ is isoquinoline, quinoline, quinazoline, cinnoline, purine, tetrahydroisoquinoline, indole, isoindole, indoline, pyridine, pyrimidine, pyridazine, pyrazine, benzimidazole, benzothiazole, benzoxazole, imidazole, pyrrole, thiazole, oxazole, isothiazole, phenyl, or naphthyl, wherein G¹ is optionally substituted 1 to 4 times with R⁵, wherein R⁵ is R^{b}.
In another embodiment of the compound of Formula (Ia), G¹ is a heterocyclyl, heteroaryl, fused cycloalkylheteroaryl, fused heterocyclylaryl, or fused heterocyclylheteroaryl ring containing at least one nitrogen atom, wherein the nitrogen atom in G¹ is ortho to the atom in G¹ connected to the remainder of the compound of Formula (Ia).
In another embodiment of the compound of Formula (Ia), G¹ is isoquinoline-3-yl, quinoline-2-yl, quinoline-3-yl, pyridine-2-yl, pyridine-3-yl, phenyl, or naphthyl-2-yl, wherein G¹ may be substituted or unsubstituted. In a further embodiment, G¹ is isoquinoline-3-yl, pyridine-2-yl, pyridine-3-yl, or phenyl, wherein G¹ may be substituted or unsubstituted. In an further embodiment, G¹ is isoquinoline-3-yl, pyridine-2-yl, or pyridine-3-yl, wherein G¹ is unsubstituted.
In another embodiment of the compound of Formula (Ia), R¹¹ and R¹² are independently selected from the group consisting of hydrogen, C₁₋₁₀ alkyl, cycloalkyl, phenyl, and —C₁₋₁₀ alkylene-phenyl. In a further embodiment, R¹¹ and R¹² are hydrogen.
In another embodiment, v is 1. In another embodiment, v is 0.
In another embodiment,
   v is 1,
   L¹ is a direct bond,
   L² is a direct bond,
   Y¹ is —N(R¹⁶)C(O)—, and
   R² is R^{f}.
In another embodiment,
   v is 1,
   L¹ is a direct bond,
   L² is a direct bond,
   R² is hydrogen, C₁₋₁₀ alkyl, cycloalkyl, phenyl, or —C₁₋₁₀ alkylene-phenyl, wherein alkyl, cycloalkyl, and phenyl are substituted or unsubstituted, and
   Y¹ is —N(R¹⁶)C(O)—, wherein R¹⁶ is hydrogen, C₁₋₁₀ alkyl, cycloalkyl, or phenyl, wherein alkyl, cycloalkyl, and phenyl are substituted or unsubstituted.
In another embodiment,
   v is 1,
   L¹ is a direct bond,
   L² is a direct bond,
   Y¹ is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S())2—, —N(R¹⁶)SO₂N(R¹⁷)—, and —C(R^{f})═C(R^{g})—;
      wherein
         R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted; and
   R² is C₁₋₁₀ alkyl, cycloalkyl, phenyl, or —C₁₋₁₀ alkylene-phenyl, wherein alkyl, cycloalkyl, and phenyl are substituted or unsubstituted.
In another embodiment,
   v is 1,
   L¹ is a direct bond,
   L² is a direct bond,
   Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted;
   R² is C₁₋₁₀ alkyl, -cycloalkyl, -phenyl, or —C₁₋₁₀ alkylene-phenyl, wherein alkyl, cycloalkyl, and phenyl are substituted or unsubstituted.
In another embodiment,
   v is 1,
   L¹ is a direct bond,
   L² is a direct bond,
   Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted; and p1 R² is methyl, ethyl, propyl, isopropyl, tert-butyl, isobutyl, 1-ethyl-propyl, and (1-halo-1-methyl)-ethyl.
In another embodiment, L³, L⁴, and L⁵ are direct bonds.
In another embodiment, L³, L⁴, and L⁵ are direct bonds, and Y² is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, and —C(R^{f})═C(R^{g})—;
   wherein
      R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.
In another embodiment, L³, L⁴, and L⁵ are direct bonds, and Y² is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
   wherein
      R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.
In another embodiment,
   L³, L⁴, and L⁵ are direct bonds,
   Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted;
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted.
In another embodiment,
   L³, L⁴, and L⁵ are direct bonds,
   Y² is selected from the group consisting of —C(O)N(R¹⁶)—, and —N(R¹⁶)C(O)—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
   p is an integer from 1 to 4.
In another embodiment,
   L³, L⁴, and L⁵ are direct bonds,
   Y² is selected from the group consisting of —C(O)N(R¹⁶)—, and —N(R¹⁶)C(O)—;
      wherein
         R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
   R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted,
   p is 1, and
   R⁶ is halo and is at the 4-position of the phenyl ring.

In the compounds of Formula (I), the various functional groups represented should be understood to have a point of attachment at the functional group having the hyphen. In other words, in the case of —C₁₋₁₀ alkylene-aryl, it should be understood that the point of attachment is the alkylene group; an example would be benzyl. In the case of a group such as —C(O)—NH—C₁₋₁₀ alkylene-aryl, the point of attachment is the carbonyl carbon.

The present invention provides antiviral compounds and compositions. As used herein “antiviral” refers to the capability of a compound of the present invention to reduce the number of viral particles in an infected subject (e.g., a cell line, a person or an animal) and/or reduce the likelihood of a subject exposed to potentially infective viral particles to contract a viral disease. In other words, the number of viral particles that infect a subject, or the likelihood of a subject to be infected by viral particles, is reduced with the administration of an antiviral compound or composition compared to that without the administration of the antiviral compound or composition. In certain embodiments, an antiviral compound or composition inhibits or reduces the contact between the viral particles and the subject, and/or the replication or emission of the viral particles.

As used herein, the term “comprises” means “includes, but is not limited to.”

Also included within the scope of the invention are the individual enantiomers of the compounds represented by Formula (I) above as well as any wholly or partially racemic mixtures thereof. The present invention also covers the individual enantiomers of the compounds represented by Formula (I) above as mixtures with diastereoisomers thereof in which one or more stereocenters are inverted. Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In another aspect, the present invention provides a pharmaceutically acceptable salt, solvate, or prodrug of compounds of Formula (I). In an embodiment, the prodrug comprises a biohydrolyzable ester or biohydrolyzable amide of a compound of Formula (I).

In another aspect, the present invention comprises a pharmaceutical composition comprising the compound of Formula (I) and a pharmaceutically acceptable carrier, excipient, diluent, or a mixture thereof. The present invention further provides uses for compounds of Formula (I) as antiviral agents such as treating viral infections in a subject or reducing the likelihood of a subject exposed to potentially infective viral particles to contract a viral disease.

Examples of compounds of Formula (I) of the present invention having potentially useful antiviral activity are listed by name below in Table 1. The ability of compounds Formula (I) to inhibit viral replication was established with representative compounds of Formula (I) listed in Table 1 using the vaccinia viral assay described in the Examples section. The compounds of Formula (I) in Table 1 were found to inhibit viral replication with an EC₅₀ of less than or equal to 100 microMolar (μM; 10⁻⁶ M). Various compounds such as Examples 1, 5, 6, 15, and 17 have an EC₅₀ of less than or equal to about 0.5 μM.

TABLE 1

| Ex. | Structure | Name |
|---|---|---|
| 1 | 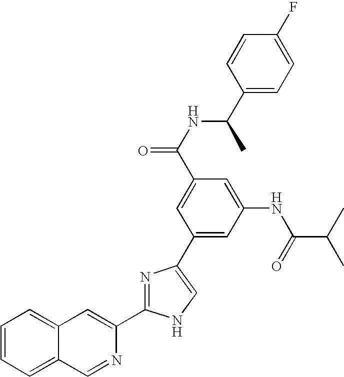 | N-[(1R)-1-(4-fluorophenyl)ethyl]-3- (2-isoquinoline-3-yl-1H-imidazol-4- yl)-5-isobutyrylamino)-benzamide |
| 2 | 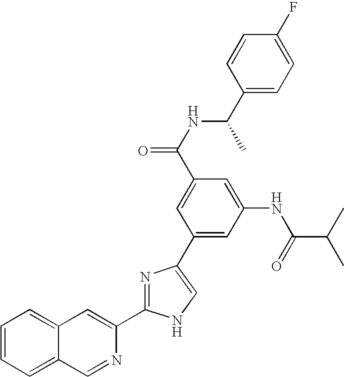 | N-[(1S)-1-(4-fluorophenyl)ethyl]-3- (2-isoquinoline-3-yl-1H-imidazol-4- yl)-5-isobutyrylamino)-benzamide |
| 3 | 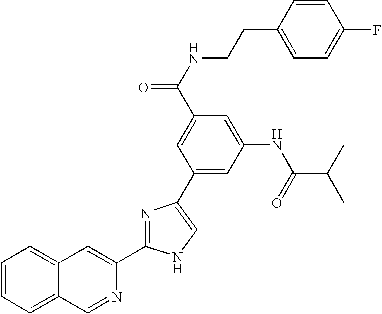 | N-[4-fluorophenethyl]-3-(2- isoquinoline-3-yl-1H-imidazol-4-yl)- 5-isobutyrylamino)-benzamide |
| 4 | 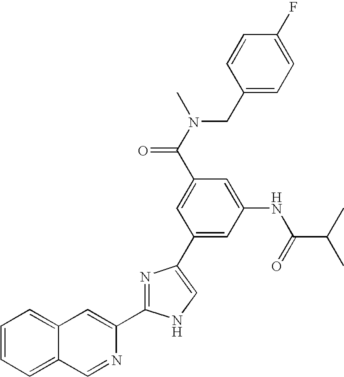 | N-(4-Fluorobenzyl)-N-methyl-3-(2- isoquinoline-3-yl-1H-imidazol-4-yl)- 5-isobutyrylamino)-benzamide |
| 5 | 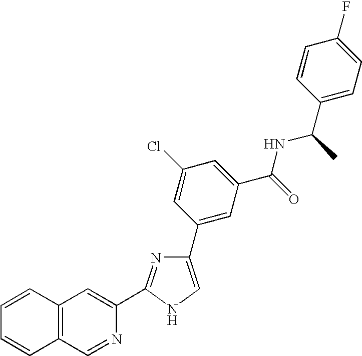 | N-[(1R)-1-(4-fluorophenyl)ethyl]-3- (2-isoquinoline-3-yl-1H-imidazol-4- yl)-5-chlorobenzamide |
| 6 | 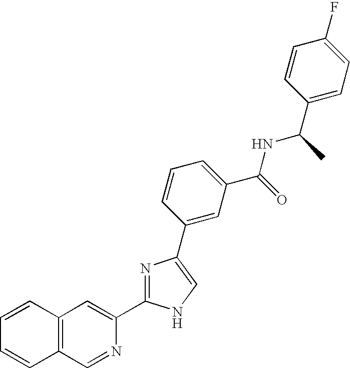 | N-[(1R)-1-(4-fluorophenyl)ethyl]-3- (2-isoquinoline-3-yl-1H-imidazol-4- yl)-benzamide |
| 7 | 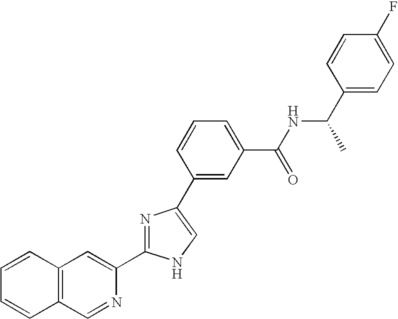 | N-[(1S)-1-(4-fluorophenyl)ethyl]-3- (2-isoquinoline-3-yl-1H-imidazol-4- yl)-benzamide |
| 8 | 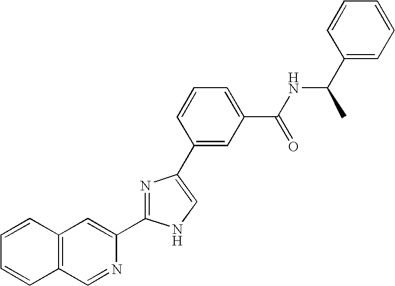 | N-[(1R)-1-phenylethyl]-3-(2- isoquinoline-3-yl-1H-imidazol-4-yl)- benzamide |
| 9 | 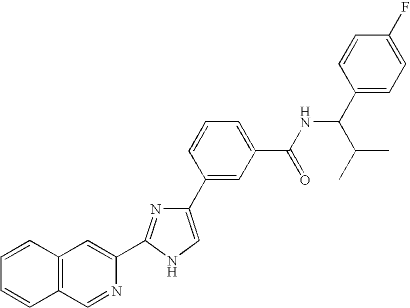 | N-[1-(4-fluorophenyl)-2- methylpropyl]-3-(2-isoquinoline-3-yl- 1H-imidazol-4-yl)-benzamide |
| 10 | 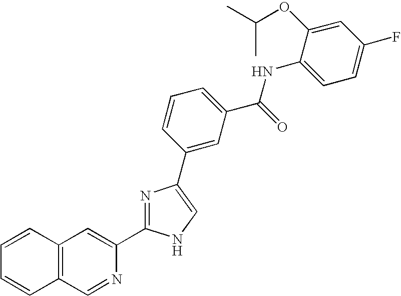 | N-(2-isoproxy-4-fluorophenyl)-3- (2-isoquinoline-3-yl-1H-imidazol-4- yl)-benzamide |
| 11 | 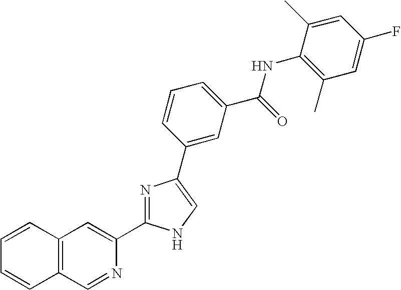 | N-(2,4-dimethyl-4-fluorophenyl)-3-(2- isoquinoline-3-yl-1H-imidazol-4-yl)- benzamide |
| 12 | 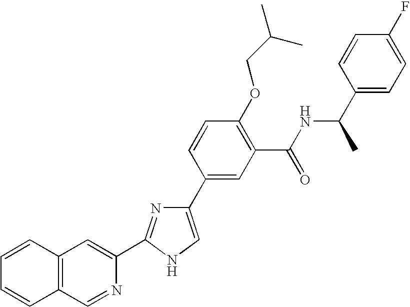 | N-[(1R)-1-(4-fluorophenyl)ethyl]-2- isobutoxy-5-(2-isoquinoline-3-yl-1H- imidazol-4-yl)-benzamide |
| 13 | 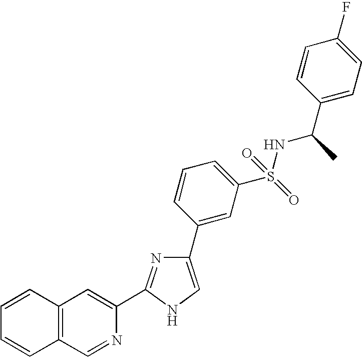 | N-[(1R)-1-(4-fluorophenyl)ethyl]-3- (2-isoquinoline-3-yl-1H-imidazol-4- yl)-benzenesulfonamide |
| 14 | 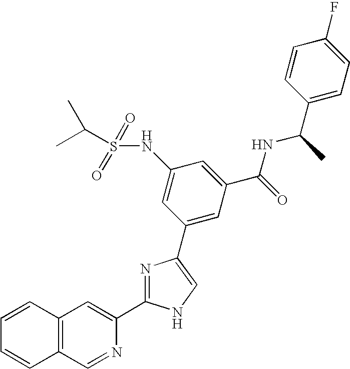 | N-[(1R)-1-(4-fluorophenyl)ethyl]-3- (2-isoquinoline-3-yl-1H-imidazol-4- yl)-5-isobutylsulfonylamino)- benzamide |
| 15 | 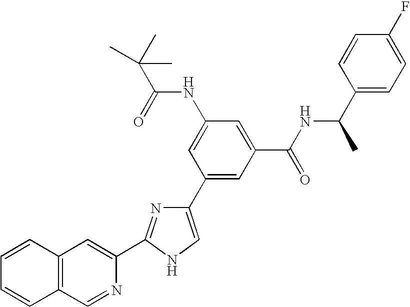 | N-[(1R)-1-(4-fluorophenyl)ethyl]-3- (2-isoquinoline-3-yl-1H-imidazol-4- yl)-5-pivaloylamino)-benzamide |
| 16 | 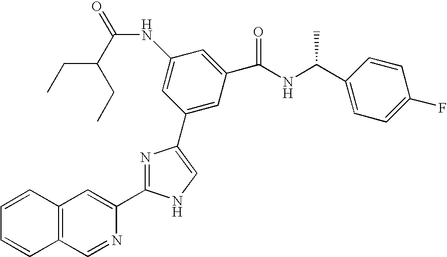 | N-[(1R)-1-(4-fluorophenyl)ethyl]-3- (2-isoquinoline-3-yl-1H-imidazol-4- yl)-5-(pentane-2-carbonylamino)- benzamide |
| 17 | 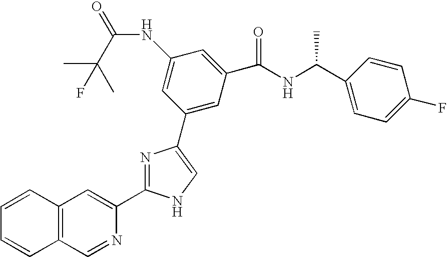 | N-[(1R)-1-(4-fluorophenyl)ethyl]-3- (2-isoquinoline-3-yl-1H-imidazol-4- yl)-5-(2-fluoroisobutyrylamino)- benzamide |
| 18 | 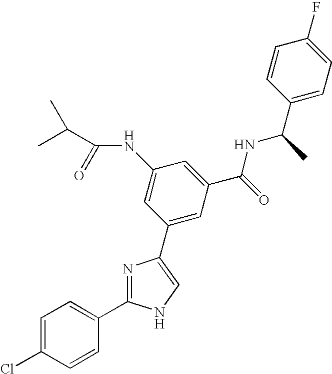 | N-[(1R)-1-(4-fluorophenyl)ethyl]-3- ((4-chlorophenyl)-1H-imidazol-4-yl)- 5-isobutyrylamino-benzamide |
| 19 | 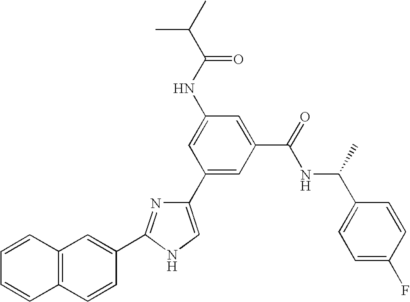 | N-[(1R)-1-(4-fluorophenyl)ethyl]-3- ((2-naphthyl)-1H-imidazol-4-yl)-5- isobutyrylamino-benzamide |
| 20 | 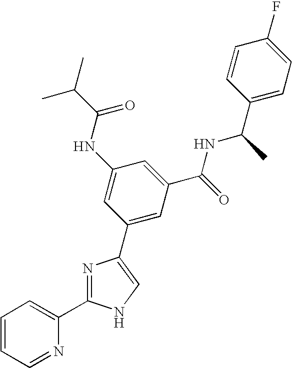 | N-[(1R)-1-(4-fluorophenyl)ethyl]-3- ((2-pyridyl)-1H-imidazol-4-yl)-5- isobutyrylamino-benzamide |
| 21 | 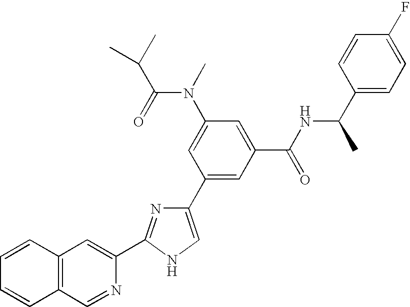 | N-[(1R)-1-(4-fluorophenyl)ethyl]-3- ((2-isoquinoline-3-yl)-1H-imidazol-4- yl)-5-isobutyrylmethylamino- benzamide |
| 22 | 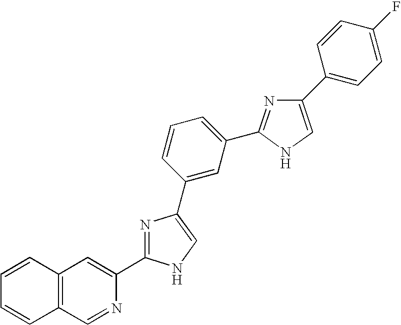 | 3-{4-[3-(4-(4-fluorophenyl)-1H- imidazol-2-yl)phenyl]-1H-imidazol-2- yl}isoquinoline |
| 23 | 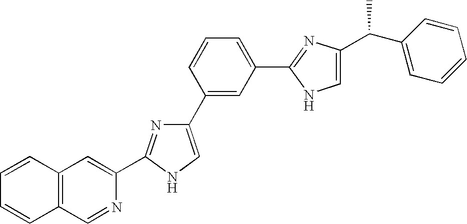 | 3-[4-(3-{4-[(1R)-1-phenylethyl]-1H- imidazol-2-yl}phenyl)-1H-imidazol-2- yl]isoquinoline |
| 24 | 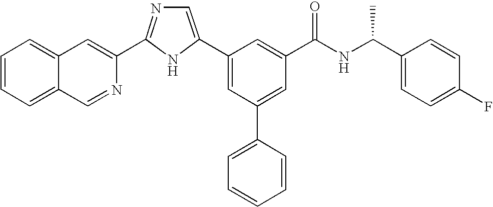 | N-[(1R)-1-(4-fluorophenyl)ethyl]-3- (2-isoquinoline-3-yl-1H-imidazol-4- yl)-5-phenylbenzamide |
| 25 | 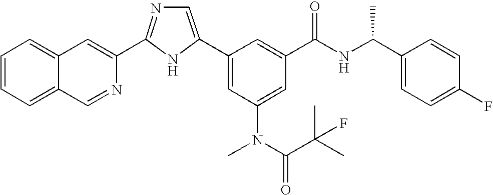 | N-[(1R)-1-(4-fluorophenyl)ethyl]-3- (2-isoquinoline-3-yl-1H-imidazol-4- yl)-5-(2-fluoroisobutyryl- methylamino)-benzamide |
| 26 | 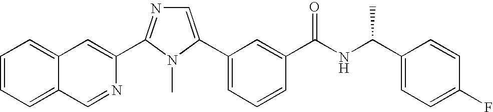 | N-[(1R)-(4-Fluoro-phenyl)-ethyl]-3- (2-isoquinoline-3-yl-3-methyl-3H- imidazol-4-yl)-benzamide |
| 27 | 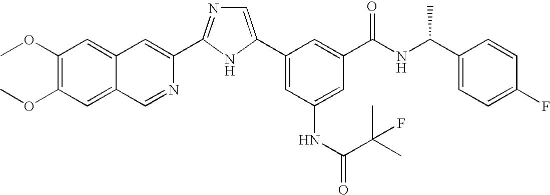 | 3-[2-(6,7-Dimethoxy-isoquinolin-3- yl)-3H-imidazol-4-yl]-5-(2-fluoro-2- methyl-propionylamino)-N-[(1R)-(4- fluoro-phenyl)-ethyl]-benzamide |
| 28 | 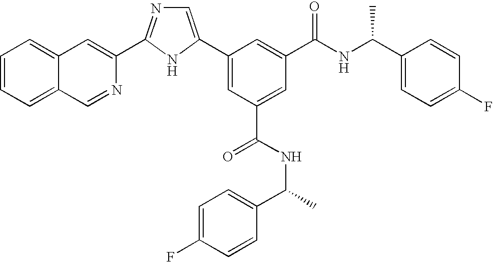 | N,N′-Bis-[(1R)-(4-fluoro-phenyl)- ethyl]-5-(2-isoquinolin-3-yl-3H- imidazol-4-yl)-isophthalamide |
| 29 | 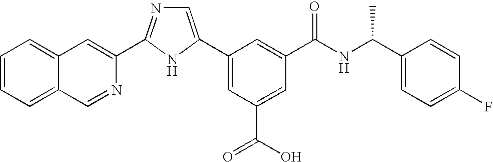 | N-[(1R)-(4-Fluoro-phenyl)-ethyl]-5- (2-isoquinolin-3-yl-3H-midazol-4-yl)- isophthalamic acid |
| 30 | 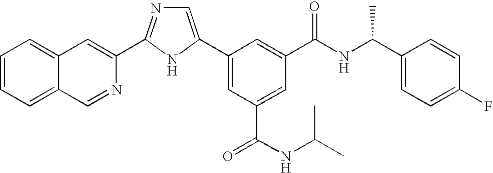 | N-[(1R)-(4-Fluoro-phenyl)-ethyl]-N′- isopropyl-5-(2-isoquinolin-3-yl-3H- imidazol-4-yl)-isophthalamide |
| 31 | 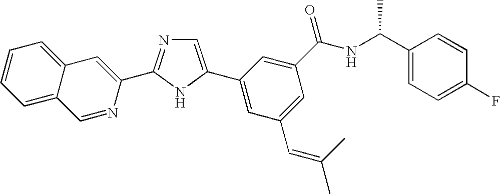 | N-[(1R)-(4-Fluoro-phenyl)-ethyl]-3- (2-isoquinolin-3-yl-3H-imidazol-4- yl)-5-(2-methyl-propenyl)-benzamide |
| 32 | 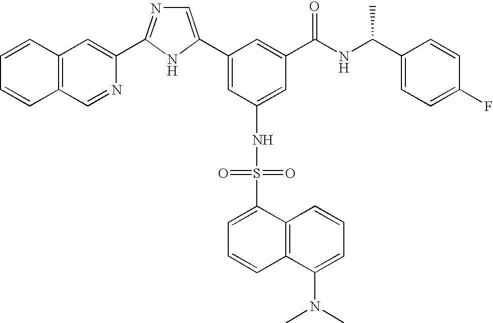 | 3-(5-Dimethylamino-naphthalene-1- sulfonylamino)-N-[(R)-1-(4-fluoro- phenyl)-ethyl]-5-(2-isoquinolin-3-yl- 3H-imidazol-4-yl)-benzamide |
| 33 | 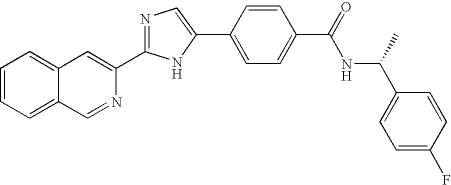 | N-[1-(4-Fluoro-phenyl)-ethyl]-4-(2- isoquinoline-3-yl-3H-imidazol-4-yl)- benzamide |
| 34 | 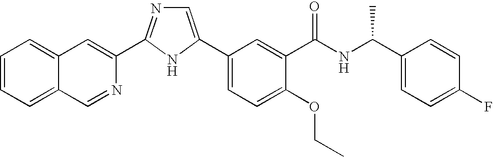 | 2-Ethoxy-N-[(1R)-(4-fluoro-phenyl)- ethyl]-5-(2-isoquinolin-3-yl-3H- imidazol-4-yl)-benzamide |
| 35 | 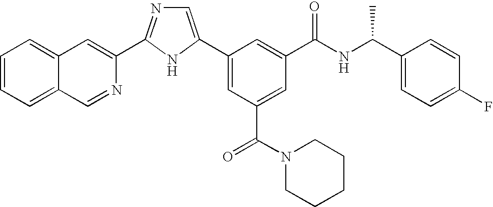 | N-[(1R)-(4-Fluoro-phenyl)-ethyl]-3- (2-isoquinoline-3-yl-3H-imidazol-4- yl)-5-(piperidine-1-carbonyl)- benzamide |
| 36 | 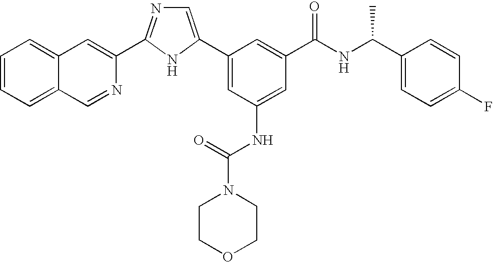 | Morpholine-4-carboxylic acid [3- [(1R)-(4-fluoro-phenyl)- ethylcarbamoyl]-5-(2-isoquinolin-3- yl-3H-imidazol-4-yl)-phenyl]-amide |
| 37 | 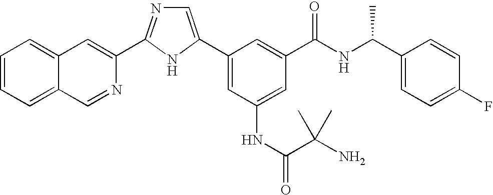 | 3-(2-Amino-2-methyl- propionylamino)-N-[(1R)-(4-fluoro phenyl)-ethyl]-5-(2-isoquinolin-3-yl- 3H-imidazol-4-yl)-benzamide |
| 38 | 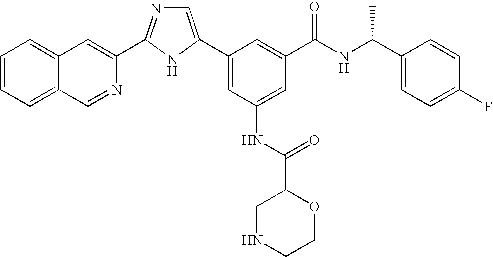 | Morpholine-2-carboxylic acid [3- [(1R)-(4-fluoro-phenyl)- ethylcarbamoyl]-5-(2-isoquinolin-3- yl-3H-imidazol-4-yl)-phenyl]-amide |
| 39 | 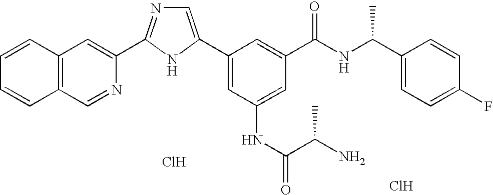 | 3-(2S-Amino-propionylamino)-N- [(1R)-(4-fluoro-phenyl)-ethyl]-5-(2- isoquinolin-3-yl-3H-imidazol-4-yl)- benzamide Dihydrochloride |
| 40 | 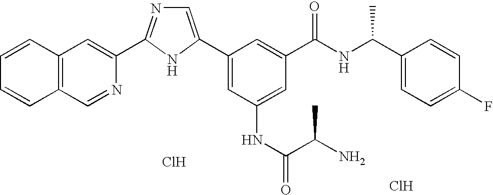 | 3-(2R-Amino-propionylamino)-N- [(1R)-(4-fluoro-phenyl)-ethyl]-5-(2- isoquinolin-3-yl-3H-imidazol-4-yl)- benzamide Dihydrochloride |
| 41 | 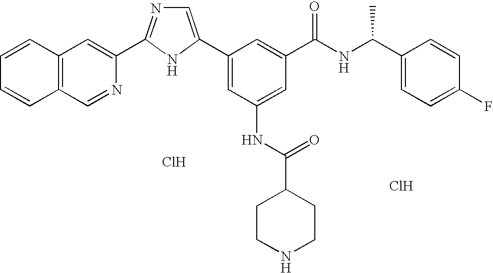 | Piperidine-4-carboxylic acid [3-[(1R)- (4-fluoro-phenyl)-ethylcarbamoyl]-5- (2-isoquinolin-3-yl-3H-imidazol-4- yl)-phenyl]-amide Dihydrochloride |
| 42 | 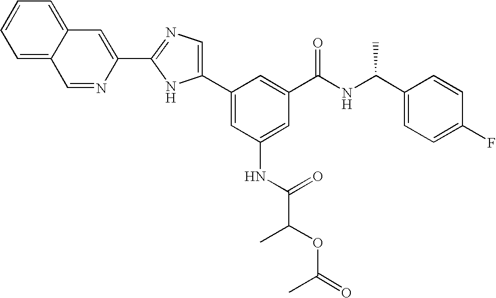 | Acetic acid 1-[3-[(1R)-(4-fluoro- phenyl)-ethylcarbamoyl]-5-(2- isoquinolin-3-yl-3H-imidazol-4-yl)- phenylcarbamoyl]-ethyl ester |
| 43 | 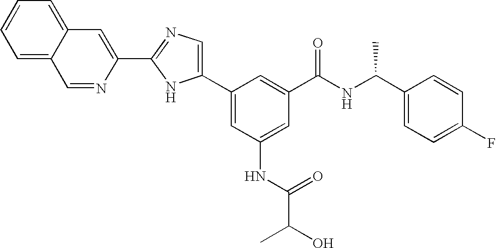 | N-[(1R)-(4-Fluoro-phenyl)-ethyl]-3- (2-hydroxy-propionylamino)-5-(2- isoquinoline-3-yl-3H-imidazol-4-yl)- benzamide |
| 44 | 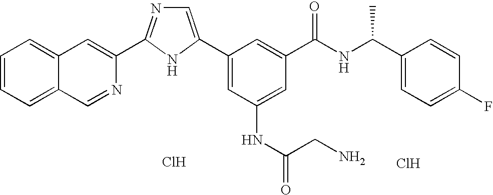 | 3-(2-Amino-acetylamino)-N-[(1R)-(4- fluoro-phenyl)-ethyl]-5-(2- isoquinolin-3-yl-3H-imidazol-4-yl)- benzamide Dihydrochloride |
| 45 | 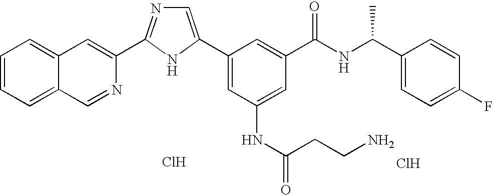 | 3-(3-Amino-propionylamino)-N- [(1R)-(4-fluoro-phenyl)-ethyl]-5-(2- isoquinolin-3-yl-3H-imidazol-4-yl)- benzamide Dihydrochloride |
| 46 | 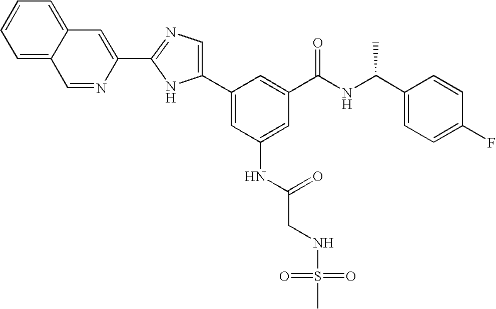 | N-[(1R)-(4-Fluoro-phenyl)-ethyl]-3- (2-isoquinolin-3-yl-3H-imidazol-4- yl)-5-(2-methanesulfonylamino- acetylamino)-benzamide |
| 47 | 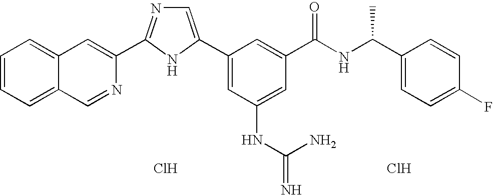 | N-[(1R)-(4-Fluoro-phenyl)-ethyl]-3- guanidino-5-(2-isoquinolin-3-yl-3H- imidazol-4-yl)-benzamide Dihydrochloride |
| 48 | 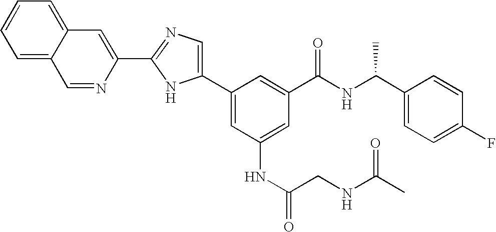 | 3-(2-Acetylamino-acetylamino)-N- [(1R)-(4-fluoro-phenyl)-ethyl]-5-(2- isoquinolin-3-yl-3H-imidazol-4-yl)- benzamide |
| 49 | 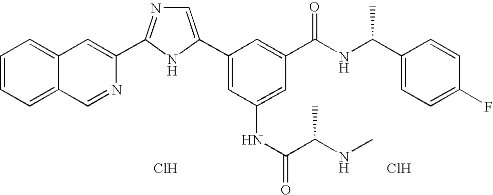 | N-[(1R)-(4-Fluoro-phenyl)-ethyl]-3- (2-isoquinolin-3-yl-3H-imidazol-4- yl)-5-(2-methylamino- propionylamino)-benzamide Dihydrochloride |
| 50 | 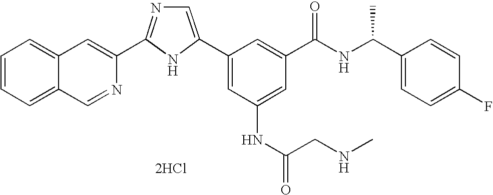 | N-[(1R)-(4-Fluoro-phenyl)-ethyl]-3- (2-isoquinolin-3-yl-3H-imidazol-4- yl)-5-(2-methylamino-acetylamino)- benzamide Dihydrochloride |
| 51 | 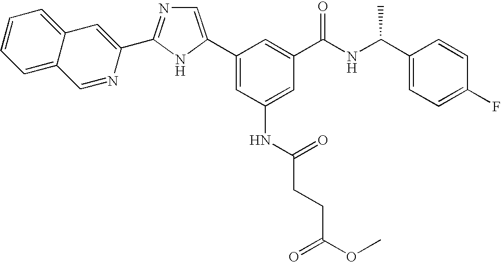 | N-[(1R)-(4-Fluoro-phenyl)- ethylcarbamoyl]-5-(2-isoquinolin-3- yl-3H-imidazol-4-yl)-phenyl]- succinamic acid methyl ester |
| 52 | 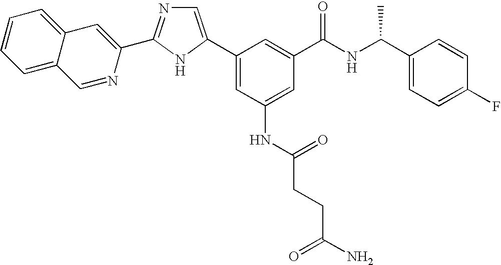 | N-[3-[(1R)-(4-Fluoro-phenyl)- ethylcarbamoyl]-5-(2-isoquinolin-3- yl-3H-imidazol-4-yl)-phenyl]- succinamide |
| 53 | 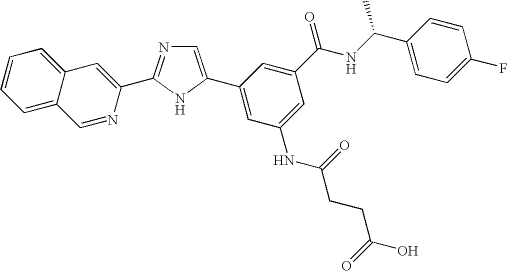 | N-[3-[1-(4-Fluoro-phenyl)- ethylcarbamoyl]-5-(2-isoquinolin-3- yl-3H-imidazol-4-yl)-phenyl]- succinamic acid |
| 54 | 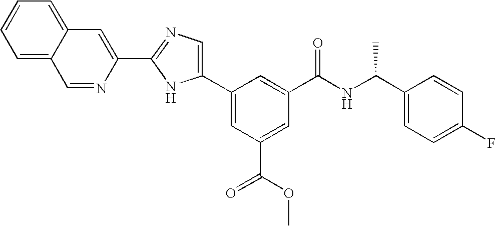 | N-[(1R)-(4-Fluoro-phenyl)-ethyl]-5- (2-isoquinolin-3-yl-3H-imidazol-4- yl)-isophthalamic acid methyl ester |

As used herein, the term “lower” refers to a group having between one and six carbons.

As used herein, the term “alkyl” refers to a straight or branched chain hydrocarbon having from one to ten carbon atoms, optionally substituted and multiple degrees of substitution being allowed. Examples of “alkyl” as used herein include, but are not limited to, methyl, N-butyl, t-butyl, N-pentyl, isobutyl, and isopropyl, and the like.

As used herein, the term “alkylene” refers to a straight or branched chain divalent hydrocarbon radical having from one to ten carbon atoms, optionally substituted and multiple degrees of substitution being allowed. Examples of “alkylene” as used herein include, but are not limited to, methylene, ethylene, and the like.

As used herein, the term “alkyline” refers to a straight or branched chain trivalent hydrocarbon radical having from one to ten carbon atoms, optionally substituted and multiple degrees of substitution being allowed. Examples of “alkyline” as used herein include, but are not limited to, methine, ethyline, and the like.

As used herein, the term “alkenyl” refers to a hydrocarbon radical having from two to ten carbons and at least one carbon-carbon double bond, optionally substituted and multiple degrees of substitution being allowed. Examples of “alkenyl” as used herein include, but are not limited to, 3,3-dimethyl-but-1-enyl, 4-hex-1-enyl, and the like.

As used herein, the term “alkenylene” refers to a straight or branched chain divalent hydrocarbon radical having from two to ten carbon atoms and one or more carbon-carbon double bonds, optionally substituted and multiple degrees of substitution being allowed. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon-carbon triple bond, optionally substituted and multiple degrees of substitution being allowed. Examples of “alkynyl” as used herein include, but are not limited to, 4-hex-1ynyl, 3,3-dimethyl-but-1ynyl, and the like.

As used herein, the term “alkynylene” refers to a straight or branched chain divalent hydrocarbon radical having from two to ten carbon atoms and one or more carbon-carbon triple bonds, optionally substituted and multiple degrees of substitution being allowed. Examples of “alkynylene” as used herein include, but are not limited to, ethyne-1,2-diyl, propyne-1,3-diyl, and the like.

As used herein, the terms “haloaliphatic”, “haloalkyl”, “haloalkenyl” and “haloalkoxy” refer to an aliphatic, alkyl, alkenyl or alkoxy group, as the case may be, substituted with one or more halogen atoms.

As used herein, “cycloalkyl” refers to a non-aromatic alicyclic hydrocarbon group and optionally possessing one or more degrees of unsaturation, having from three to twelve carbon atoms, optionally substituted and multiple degrees of substitution being allowed. Examples of “cycloalkyl” as used herein include, but are not limited to, cyclopropyl, cyclobutyl, cyclopentyl, cyclohexyl, cycloheptyl, cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents and multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, cyclooctyl-1,5-diyl, and the like.

As used herein, the term “heterocyclic” or the term “heterocyclyl” refers to a non-aromatic three to twelve-membered heterocyclic ring optionally possessing one or more degrees of unsaturation, containing one or more heteroatomic substitutions selected from S, SO, SO₂, O, or N, optionally substituted and multiple degrees of substitution being allowed. Such a ring may be optionally fused to from one to three of another “heterocyclic” ring(s) or cycloalkyl ring(s). Examples of “heterocyclyl” include, but are not limited to, tetrahydrofuran, 1,4-dioxane, 1,3-dioxane, piperidine, pyrrolidine, morpholine, piperazine, and the like.

As used herein, the term “heterocyclylene” refers to a non-aromatic three to twelve-membered heterocyclic ring diradical optionally having one or more degrees of unsaturation containing one or more heteroatoms selected from S, SO, SO₂, O, or N, optionally substituted and multiple degrees of substitution being allowed. Such a ring may be optionally fused to from one to three benzene rings or to one to three of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

As used herein, the term “aryl” refers to a benzene ring or to benzene ring fused to one to three benzene rings, optionally substituted and multiple degrees of substitution being allowed. Examples of aryl include, but are not limited to, phenyl, 2-Naphthyl, 1-naphthyl, 1-anthracenyl, and the like.

As used herein, the term “arylene” refers to a benzene ring diradical or to a benzene ring system diradical fused to one to three optionally substituted benzene rings, optionally substituted and multiple degrees of substitution being allowed. Examples of “arylene” include, but are not limited to, benzene-1,4-diyl, naphthalene-1,8-diyl, and the like.

As used herein, the term “heteroaryl” refers to a five-to seven-membered aromatic ring, or to a polycyclic (up to three rings) aromatic ring, containing one or more nitrogen, oxygen, or sulfur heteroatoms, where N-Oxides and sulfur monoxides and sulfur dioxides are permissible heteroaromatic substitutions, optionally substituted and multiple degrees of substitution being allowed. For polycyclic heteroaryl aromatic ring systems, one or more of the rings may contain one or more heteroatoms. Examples of “heteroaryl” used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five- to seven-membered aromatic ring diradical, or to a polycyclic (up to three rings) heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted and multiple degrees of substitution being allowed. For polycyclic aromatic ring system diradicals, one or more of the rings may contain one or more heteroatoms. Examples of “heteroarylene” used herein include, but are not limited to, furan-2,5-diyl, thiophene-2,4-diyl, 1,3,4-Oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “fused cycloalkylaryl” refers to one or two cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl, 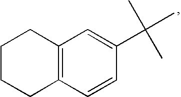
and the like.

As used herein, the term “fused cycloalkylarylene” refers to a fused cycloalkylaryl, wherein the aryl group is divalent. Examples include 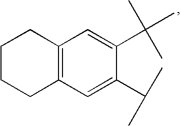
and the like.

As used herein, the term “fused arylcycloalkyl” refers to one or two aryl groups fused to a cycloalkyl group, the cycloalkyl and aryl groups having two atoms in common, and wherein the cycloalkyl group is the point of substitution. Examples of “fused arylcycloalkyl” used herein include 1-indanyl, 2-indanyl, 9-fluorenyl, 1-(1,2,3,4-tetrahydronaphthyl), 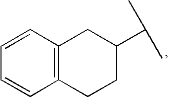
and the like.

As used herein, the term “fused arylcycloalkylene” refers to a fused arylcycloalkyl, wherein the cycloalkyl group is divalent. Examples include 9,1-fluorenylene, 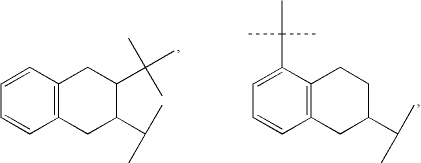
and the like.

As used herein, the term “fused heterocyclylaryl” refers to one or two heterocyclyl groups fused to an aryl group, the aryl and heterocyclyl groups having two atoms in common, and wherein the aryl group is the point of substitution. Examples of “fused heterocyclylaryl” used herein include 3,4-methylenedioxy-1-phenyl, 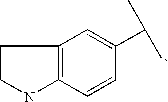
and the like

As used herein, the term “fused heterocyclylarylene” refers to a fused heterocyclylaryl, wherein the aryl group is divalent. Examples include 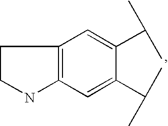
and the like.

As used herein, the term “fused arylheterocyclyl” refers to one or two aryl groups fused to a heterocyclyl group, the heterocyclyl and aryl groups having two atoms in common, and wherein the heterocyclyl group is the point of substitution. Examples of “fused arylheterocyclyl” used herein include 2-(1,3-benzodioxolyl), 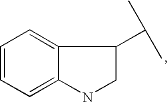
and the like.

As used herein, the term “fused arylheterocyclylene” refers to a fused arylheterocyclyl, wherein the heterocyclyl group is divalent. Examples include 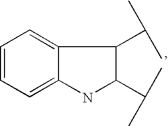
and the like.

As used herein, the term “fused cycloalkylheteroaryl” refers to one or two cycloalkyl groups fused to a heteroaryl group, the heteroaryl and cycloalkyl groups having two atoms in common, and wherein the heteroaryl group is the point of substitution. Examples of “fused cycloalkylheteroaryl” used herein include 5-aza-6-indanyl, 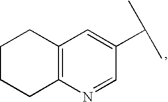
and the like.

As used herein, the term “fused cycloalkylheteroarylene” refers to a fused cycloalkylheteroaryl, wherein the heteroaryl group is divalent. Examples include 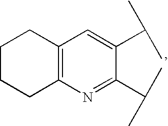
and the like.

As used herein, the term “fused heteroarylcycloalkyl” refers to one or two heteroaryl groups fused to a cycloalkyl group, the cycloalkyl and heteroaryl groups having two atoms in common, and wherein the cycloalkyl group is the point of substitution. Examples of “fused heteroarylcycloalkyl” used herein include 5-aza-1-indanyl, 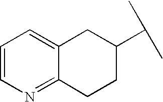
and the like.

As used herein, the term “fused heteroarylcycloalkylene” refers to a fused heteroarylcycloalkyl, wherein the cycloalkyl group is divalent. Examples include 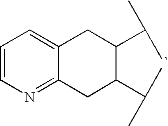
and the like.

As used herein, the term “fused heterocyclylheteroaryl” refers to one or two heterocyclyl groups fused to a heteroaryl group, the heteroaryl and heterocyclyl groups having two atoms in common, and wherein the heteroaryl group is the point of substitution. Examples of “fused heterocyclylheteroaryl” used herein include 1,2,3,4-tetrahydro-beta-carbolin-8-yl, 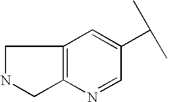
and the like.

As used herein, the term “fused heterocyclylheteroarylene” refers to a fused heterocyclylheteroaryl, wherein the heteroaryl group is divalent. Examples include 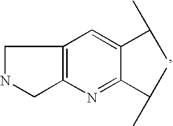
and the like.

As used herein, the term “fused heteroarylheterocyclyl” refers to one or two heteroaryl groups fused to a heterocyclyl group, the heterocyclyl and heteroaryl groups having two atoms in common, and wherein the heterocyclyl group is the point of substitution. Examples of “fused heteroarylheterocyclyl” used herein include-5-aza-2,3-dihydrobenzofuran-2-yl, 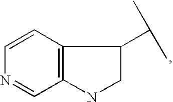
and the like.

As used herein, the term “fused heteroarylheterocyclylene” refers to a fused heteroarylheterocyclyl, wherein the heterocyclyl group is divalent. Examples include 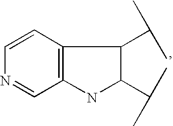
and the like.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfinyl” refers to the group RₐS(O)—, where Rₐ is alkyl.

As used herein, the term “alkenylsulfinyl” refers to the group RₐS(O)—, where Rₐ is alkenyl.

As used herein, the term “alkynylsulfinyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “aroyloxy” refers to the group RₐC(O)O—, where Rₐ is aryl.

As used herein, the term “heteroaroyloxy” refers to the group RₐC(O)O—, where Rₐ is heteroaryl.

As used herein, the term “optionally” means that the subsequently described event(s) may or may not occur, and includes both event(s) which occur and events that do not occur.

As used herein, the term “substituted” refers to substitution of one or more hydrogens of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week, or a compound which maintains its integrity long enough to be useful for therapeutic or prophylactic administration to a patient. The phrase “one or more substituents”, as used herein, refers to a number of substituents that equals from one to the maximum number of substituents possible based on the number of available bonding sites, provided that the above conditions of stability and chemical feasibility are met.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” refers iodine, bromine, chlorine or fluorine.

As used herein, the term “mercapto” refers to the substituent —SH.

As used herein, the term “carboxy” refers to the substituent —COOH.

As used herein, the term “cyano” refers to the substituent —CN.

As used herein, the term “aminosulfonyl” refers to the substituent —SO₂NH₂.

As used herein, the term “carbamoyl” refers to the substituent —C(O)NH₂.

As used herein, the term “sulfanyl” refers to the substituent —S—.

As used herein, the term “sulfinyl” refers to the substituent —S(O)—.

As used herein, the term “sulfonyl” refers to the substituent —S(O)₂—.

The compounds can be prepared according to the following reaction Schemes (in which variables are as defined before or are defined) using readily available starting materials, and reagents. In these reactions, it is also possible to make use of variants which are themselves known to those of ordinary skill in this art, but are not mentioned in greater detail.

The present invention also provides a method for the synthesis of compounds useful as intermediates in the preparation of compounds of Formula (I) along with methods for the preparation of compounds of Formula (I). Unless otherwise specified, structural variables are as defined for Formula (I).
Scheme 1 Describes a Synthesis of a Compound of Formulae (Ia), (Ib), and (Ic).

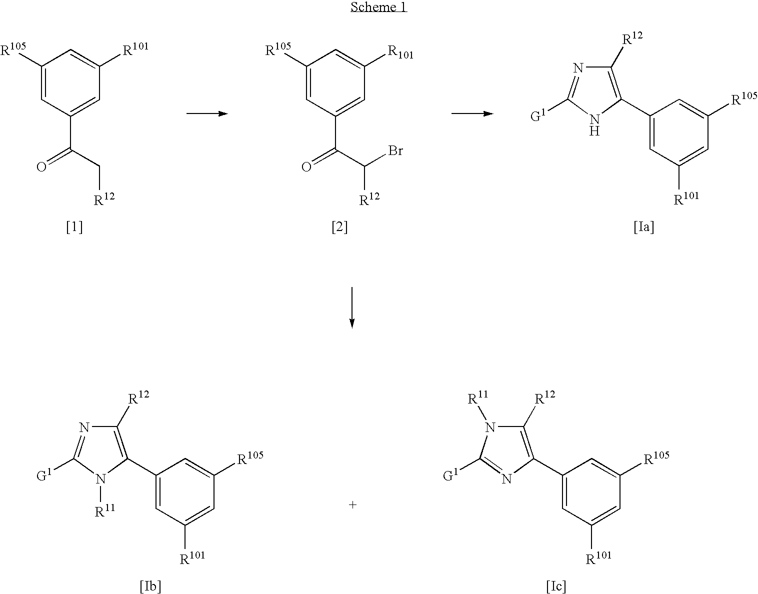

R¹⁰⁵ and R¹⁰¹ may be a group such as but not limited to aryl, heteroaryl, arylalkyl, heteroarylalkyl, alkoxycarbonyl, arylalkyloxycarbonyl, heteroalkyloxycarbonyl, arylalkylcarbamoyl, dialkylcarbamoyl, or di(arylalkyl)carbamoyl.

A ketone of formula [1] is treated with a reagent such as pyrrolidinone hydrotribromide in a solvent such as dioxane, at a temperature of from 25° C. to 125° C., to afford the bromoketone [2]. Ketone [2] may be treated with a carboxylic acid G¹-CO₂H in the presence of a reagent such as potassium carbonate in a solvent such as DMF or dioxane to afford the alkylation product, the carboxy ketone, which may be treated with ammonium acetate in acetic acid, in the presence or absence of a cosolvent such as THF or dioxane, at a temperature of from 50° C. to 180° C., to afford [Ia]. The compound of formula [Ia] may be alkylated with an alkyl halide such as R¹¹—Br in the presence of a base such as potassium carbonate, in a solvent such as DMF, to afford [Ib] and/or [Ic], where R¹¹ is alkyl. Alternately, the above alkylation product of G¹-CO₂H and [2] may be treated with an amine H₂N—R¹¹ in acetic acid and a cosolvent such as dioxane, followed by addition of ammonium acetate and heating at a temperature of from 25° C. to 180° C., to afford [Ib] and/or [Ic], with the composition of the mixture varying from 1:1 to in excess of 95:5 or 5:95, depending on the substituent nature of R¹².

Scheme 2 Describes the Synthesis of the Intermediate of Formula [1].

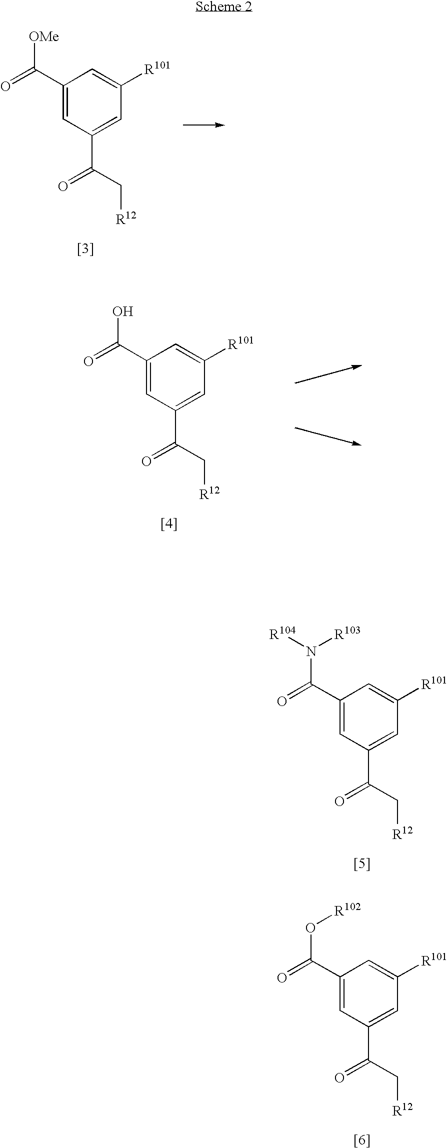

R¹⁰² and R¹⁰³ may be a group such as but not limited to aryl, heteroaryl, alkylaryl, or alkylheteroaryl.

R¹⁰⁴ may be a group such as but not limited to hydrogen, aryl, heteroaryl, alkylaryl, or alkylheteroaryl.

The ester [3] may be treated with a base such a sodium hydroxide or lithium hydroxide, in a solvent such as THF/methanol/water, at a temperature of from 0° C. to 100° C., to afford, after mild acidification, the acid [4]. Compound [4] may be treated with an amine R¹⁰³—NH—R¹⁰⁴ in the presence or absence of a base such as DIEA, in the presence of a coupling agent such as HBTU or EDC, in a solvent such as THF or DMF, at a temperature of from 0° C. to 25° C., to afford [5]. Compound [4] may also be treated with an alcohol R¹⁰²—OH in the presence or absence of a base such as DIEA, in the presence of a coupling agent such as EDC, in the presence of DMAP, in a solvent such as THF or DMF, at a temperature of from 0° C. to 25° C., to afford [6].
Scheme 3 Describes the Synthesis of Intermediates of Formula [3].

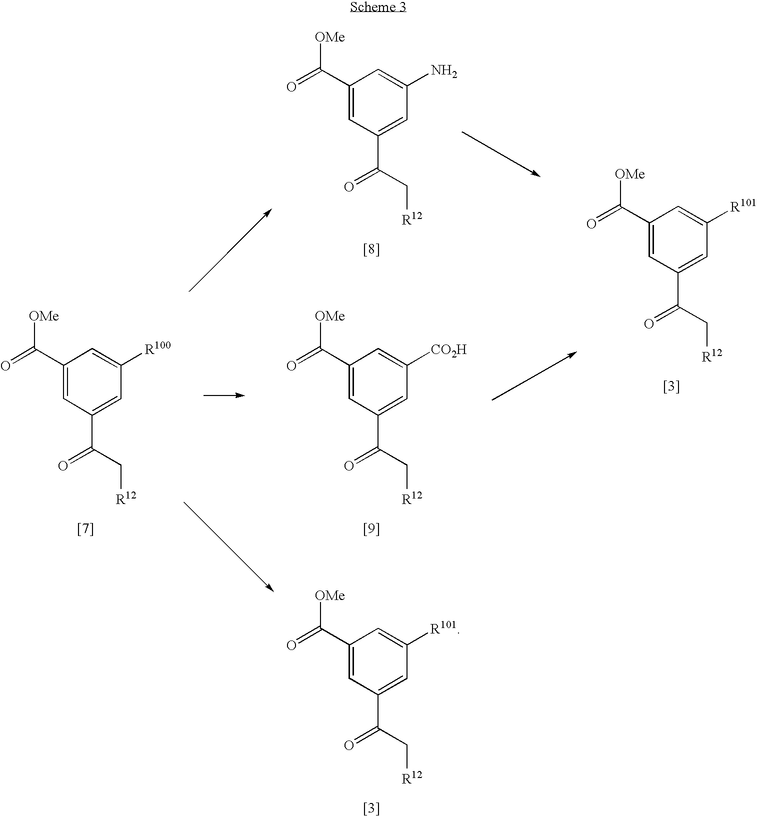

The ketone [7] where R¹⁰⁰ is —NO₂ may be treated with a reducing agent such as SnCl₂ in a solvent such as methanol or methanol-HCl aq, at a temperature of from 0° C. to 100° C., to afford the aniline [8]. Aniline [8] may be coupled with an acid, such as but not limited to an arylcarboxylic acid or (un)substituted alkyl carboxylic acid R¹⁰⁵—CO₂H in a solvent such as DMF employing EDC or HBTU, to afford [3] where R¹⁰¹ has the meaning —NHC(O)—R¹⁰⁵. Ketone [7], where R¹⁰⁰ is a group such as —CO₂-tBu, may be treated with an acid such as TFA in a solvent such as CH₂Cl₂ to afford the acid [9]. Acid [9] may be coupled with an aniline or (un)substituted alkylamine R¹⁰⁵—NH₂ in the presence or absence of a base such as DIEA, in a solvent such as THF or DMF, in the presence of a coupling agent such as HBTU or EDC, to afford [3] where R¹⁰¹ has the meaning —C(O)NH—R¹⁰⁵. Ketone [7] where R¹⁰⁰ is a group such as —Br or —I may also be treated with a reagent such as an arylboronic acid, in the presence of aqueous sodium carbonate, in the presence or absence of a cosolvent such as DME, dioxane, or THF, in the presence of a catalyst such as Pd(PPh₃)₄, at a temperature of from 25° C. to 120° C., to provide [3] where R¹⁰¹ is aryl.
Scheme 4 Describes the Synthesis of a Compound of Formula [7] where R¹⁰⁰ may be a Group such as —NO₂ or Alkoxycarbonyl.

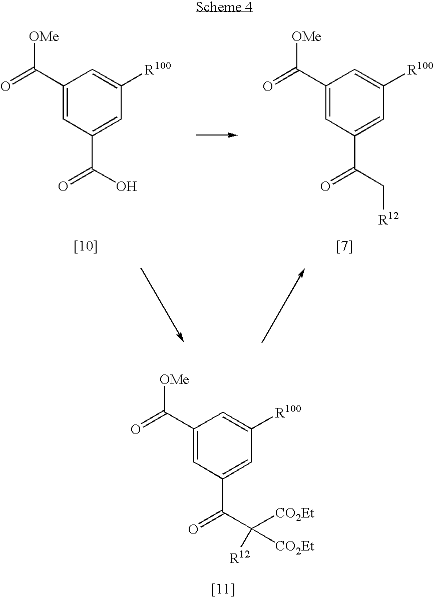

A carboxylic acid of formula [10] may be treated with a reagent such as thionyl chloride or oxalyl chloride, in a solvent such as dichloromethane, at a temperature of from 0° C. to 50° C., to afford the acid chloride. The acid chloride may be treated with the reagent formed by the combination of R¹²—CH₂MgCl and ZnCl₂ in THF, in the presence of Pd(PPh₃)₄, at a temperature of from −78° C. to 25° C., to afford [7]. Alternately, the acid chloride may be treated with the reagent formed by treatment of EtO₂C—CH(R¹²)—CO₂Et and magnesium ethoxide in a solvent such as THF or ethanol. The product ketodiester [11] may be treated in acetic acid with water and sulfuric acid and heated at a temperature of from 80° C. to 130° C. to provide the ketone with the methyl ester hydrolyzed to the acid. This material may be methylated by treatment in methanol with an acid catalyst such as sulfuric acid or HCl in dioxane at reflux, to provide the ketone [7]. Alternately, treatment of the acid with methyl iodide and a base such as potassium carbonate in a solvent such as DMF provides the ester [7].

The compounds of the present invention set forth in the present examples were found to have EC50's of less than or equal to 100 μM in the cellular based assay described below. Various compounds described below were found to have an EC50 of less than 0.5 μM in the cellular based assay described below

In general, compounds of the present invention useful for pharmaceutical applications may have EC50's of below about 10 μM. In an embodiment, embodiments of the present invention useful for pharmaceutical applications may have EC50's of below about 1 μM. For particular applications, lower inhibitory potencies may be useful. Thus, in another embodiment, compounds of the present invention may act as an antiviral with an EC₅₀ in a range of about 0.001 μM to about 1 μM.

In another embodiment, the present invention provides a pharmaceutical compositions useful as an antiviral agent, wherein the pharmaceutical composition comprises a therapeutically effective amount of a compound of Formula (I), defined above, as a single or polymorphic crystalline form or forms, an amorphous form, a single enantiomer, a racemic mixture, a single stereoisomer, a mixture of stereoisomers, a single diastereoisomer, a mixture of diastereoisomers, an isotopically enriched form, a solvate, a pharmaceutically acceptable salt, a solvate, a prodrug, a biohydrolyzable ester, or a biohydrolyzable amide thereof. The pharmaceutical composition may further comprise a pharmaceutically acceptable carrier, diluent, excipient, or a mixture thereof.

As used herein the terms “pharmaceutically acceptable carrier”, “pharmaceutically acceptable diluent”, and “pharmaceutically acceptable excipient” means the carrier, diluent or excipient must be compatible with the other ingredients of the formulation and not deleterious to the recipient thereof.

As used herein the term “therapeutically effective amount” as used herein means that amount of active compound or pharmaceutical agent that elicits the biological or medicinal response in a tissue, system, or subject that is being sought by a researcher, veterinarian, medical doctor or other clinician, which includes reducing the number of viral particles in an infected subject (e.g., a cell line, a person or an animal) and/or reducing the likelihood of a subject exposed to potentially infective viral particles to contract a viral disease. When the active compound (i.e., active ingredient) is administered as the salt, references to the amount of active ingredient are to the free acid or free base form of the compound. It should also be understood that a specific dosage and treatment regimen for any particular subject will depend upon a variety of factors, including the activity of the specific compound employed, the age, body weight, general health, sex, and diet of the patient, time of administration, rate of excretion, drug combinations, the judgment of the treating physician, and the severity of the particular disease being treated. In an embodiment, a therapeutically effective amount of the compound of Formula (I) comprises an amount sufficient to achieve and maintain a sustained blood level that at least partially inhibits viral growth.

Pharmaceutical compositions of the present invention comprising a compound of Formula (I) may be used to treat a viral condition associated with a DNA virus such as, but are not limited to, Adenoviridae including adenovirus, Hepadnaviridae including hepatitis B virus (HBV), Herpesviridae including herpes simplex virus type 1 (HSV-1), type 2 (HSV-2), thymidine kinase-deficient (TK⁻) HSV-1, varicella-zoster virus (TK⁺ and TK⁻ VZV), cytomegalovirus (CMV), human herpesvirus type 6 (HHV-6), and feline herpesvirus, Poxviridae including vaccinia virus, Papillomaviridae including human papilloma virus, and Polyomaviridae including polyoma virus.

Pharmaceutical compositions of the present invention comprising a compound of Formula (I) may be used to treat a viral condition associated with an RNA virus such as, but are not limited to, Retroviridae including human immunodeficiency virus type 1 (HIV-1) and type 2 (HIV-2), simian immunodeficiency virus (SIV), and moloney murine sarcoma virus, Coronaviridae including feline (FIPV) corona virus, human (SARS) CoV, and mouse hepatitis virus, Flaviviridae including flavivirus (yellow fever virus (YFV), dengue-type 2 virus, and modoc virus (murine flavivirus)), hepacivirus (hepatitis C, hepatitis B, hepatitis A), and pestivirus (bovine viral diarrhea virus (BVDV)), Picornaviridae including coxsackie B virus, polio virus, and rhinovirus, Alphaviridae including sindbis virus, Arenaviridae including arenaviruses (Tacaribe), Bunyaviridae including punta toro, Orthomyxoviridae including influenza A, B, and C virus, Paramyxoviridae including respiratory syncytial virus (RSV) and parainfluenza-3 virus, and Reoviridae including reo-1 virus.

Thus, in one embodiment, a therapeutically effective amount of the compounds of Formula (I) is an amount sufficient to reduce viral load in a subject. In an embodiment, the virus is an orthopox virus. For example, the compounds of the present invention may be used to inhibit smallpox infection.

In yet another embodiment, the present invention also provides a pharmaceutical composition comprising a therapeutically effective amount of the compound of Formula (I), further comprising one or more additional therapeutic agents. Additional therapeutic agents may be those as described below, or may include other therapeutic agents as may be known in the art useful to treat or reduce risk of viral infection.

As used herein, a “subject” includes, but is not limited to, a cell line, a tissue, an organ, a bird, a mammal such as a horse, cow, sheep, pig, mouse, dog, cat, or a primate such as a chimpanzee, gorilla, rhesus monkey, or human. In an embodiment, a subject is a human. In another embodiment, a subject may include one that either suffers from one or more aforesaid viral infections, or one that is at risk for contracting one or more aforesaid viral infections.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alchol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

Formulations suitable for nasal or inhalational administration wherein the carrier is a solid include a powder having a particle size for example in the range 1 to 500 microns (including particle sizes in a range between 20 and 500 microns in increments of 5 microns such as 30 microns, 35 microns, etc). Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalation therapy is readily administered by metered dose inhalers.

Formulations suitable for vaginal administration may be presented as pessaries, tampons, creams, gels, pastes, foams or spray formulations containing in addition to the active ingredient such carrier as are known in the art to be appropriate.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Also provided by the present invention are prodrugs of the invention. Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19.

Other salts which are not pharmaceutically acceptable may be useful in the preparation of compounds of the invention and these form a further aspect of the invention.

In addition, some of the compounds of the present invention may form solvates with water or common organic solvents. Such solvates are also encompassed within the scope of the invention.

Thus, in a further embodiment, there is provided a pharmaceutical composition comprising a compound of the present invention, or a pharmaceutically acceptable salt, solvate, or prodrug thereof, and a pharmaceutically acceptable carrier, excipient, diluent, or a mixture thereof.

In another aspect, the present invention provides a method of treating a viral condition comprising administering to a subject in need thereof a therapeutically effective amount of a compound of Formula (I). The compound of Formula (I) may be administered as a single or polymorphic crystalline form or forms, an amorphous form, a single enantiomer, a racemic mixture, a single stereoisomer, a mixture of stereoisomers, a single diastereoisomer, a mixture of diastereoisomers, an isotopically enriched form, a solvate, a pharmaceutically acceptable salt, a solvate, a prodrug, a biohydrolyzable ester, or a biohydrolyzable amide thereof. Further, the compounds of Formula (I) may be administered as part of a pharmaceutical composition as described above. The method of treating a viral condition may comprise administering a compound of Formula (I) or a pharmaceutical composition comprising a compound of Formula (I) to a subject prophylactically, or prior to the onset of or diagnosis of a viral infection.

In an embodiment, the present invention provides a method of treating a viral condition associated with a DNA virus such as, but not limited to, Adenoviridae including adenovirus, Hepadnaviridae including hepatitis B virus (HBV), Herpesviridae including herpes simplex virus type 1 (HSV-1), type 2 (HSV-2), thymidine kinase-deficient (TK⁻) HSV-1, varicella-zoster virus (TK⁺ and TK⁻ VZV), cytomegalovirus (CMV), human herpesvirus type 6 (HHV-6), and feline herpesvirus, Poxviridae including vaccinia virus, Papillomaviridae including human papilloma virus, and Polyomaviridae including polyoma virus, comprising administering a therapeutically effective amount of a compound of Formula (I) to a subject in need thereof.

In another embodiment, the present invention provides a method of treating a viral condition associated with an RNA virus such as, but not limited to, Retroviridae including human immunodeficiency virus type 1 (HIV-1) and type 2 (HIV-2), simian immunodeficiency virus (SIV), and moloney murine sarcoma virus, Coronaviridae including feline (FIPV) corona virus, human (SARS) CoV, and mouse hepatitis virus, Flaviviridae including flavivirus (yellow fever virus (YFV), dengue-type 2 virus, and modoc virus (murine flavivirus)), hepacivirus (hepatitis A, B, or C), and pestivirus (bovine viral diarrhea virus (BVDV)), Picornaviridae including coxsackie B virus, polio virus, and rhinovirus, Alphaviridae including sindbis virus, Arenaviridae including arenaviruses (Tacaribe), Bunyaviridae including punta toro, Orthomyxoviridae including influenza A, B, and C virus, Paramyxoviridae including respiratory syncytial virus (RSV) and parainfluenza-3 virus, and Reoviridae including reo-1 virus, comprising administering a therapeutically effective amount of a compound of Formula (I) to a subject in need thereof.

The dosage at which the compounds of Formula (I) are used may be varied depending upon the condition being treated, the size of the individual, pharmacokinetic parameters, and the individual compound. In one embodiment, the compound of Formula (I) may comprise a dosage such that the concentration of the compound of Formula (I) at the surface of a virus infected cell is about 100 micromolar (μM) or less. In another embodiment, the compound of Formula (I) may comprise a dosage such that the concentration of compound at the surface of a virus infected cell is about 50 micromolar (μM) or less. In yet another embodiment, the compound of Formula (I) may comprise a dosage such that the concentration of compound at the surface of a virus infected cell is about 10 micromolar (μM) or less.

The pharmaceutical compositions of the present invention may be administered in a form and/or route appropriate to the condition to be treated, suitable forms and routes include oral, rectal, nasal, topical (including ocular, buccal and sublingual), vaginal or parenteral (including subcutaneous, intramuscular, intravenous, intradermal, intrathecal and epidural) dosage. Generally, the compounds of this invention may be administered orally, but if an embodiment is not sufficiently orally bioavailable it can be administered by any of the other routes noted above.

In various embodiments, a compound of Formula (I) may be administered as a dose of less than 1,000 mg/kg of body weight per day, or as a dose of less than 100 mg/kg of body weight per day, or as a dose of less than 10 mg/kg of body weight per day.

The amount of active ingredient that may be combined with the carrier materials to produce a single dosage will vary depending upon the host treated and the particular mode of administration. For example, a formulation intended for oral administration to humans may contain 1 mg to 2 grams of a compound of Formula (I) with an appropriate and convenient amount of carrier material that may vary from about 5 to 95 percent of the total composition. Dosage unit forms will generally contain between from about 5 mg to about 500 mg of active ingredient. The dosage may be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

The term “treatment of a viral condition” as used herein, refers to reducing the number of viral particles in an infected subject (e.g., a cell line, tissue, organ, a person or an animal) and/or reducing the likelihood of a subject exposed to potentially infective viral particles to contract a viral disease.

As described above, the compound of Formula (I) may be used alone, or to replace or supplement a compound used to treat a viral condition. Additionally, the compound of Formula I may be used in conjunction with one or more other therapeutic agents used to treat conditions associated with a viral infection in a subject. The following is a non-exhaustive listing of adjuvants and additional therapeutic agents that may be used in combination with an antiviral agent of the present invention:
1. Analgesics: Aspirin
2. NSAIDs (Nonsteroidal anti-inflammatory drugs): Ibuprofen, Naproxen, Diclofenac
3. DMARDs (Disease-Modifying Antirheumatic drugs): Methotrexate, gold preparations, hydroxychloroquine, sulfasalazine
4. Biologic Response Modifiers, DMARDs: Etanercept, Infliximab
5. Glucocorticoids
6. Immunosuppresants and immunomodulators
   Pharmacologic classifications of treatment for bacterial or viral infection
1. gyrase inhibitors; ciprofloxacin
2. beta lactam antibiotics; cefuroxime, amoxicillin, cephalexin, ceclor, meropenem, aztreonam
3. miscellaneous antibiotics; linezolid, erythromycin, streptomycin, vancomycin, doxycycline, rifampin, isoniazid
4. antifungal agents; terbinafine, fluconazole, ketoconazole, amphotericin B, griseofulvin
5. antiviral agents
   a. Antiviral agents for AIDS treatment; AZT, abacavir, ddC, ddI, d4T, 3TC, ZDV, tenofovir, nevirapine, pentafuside, amprenavir, fosamprenavir, indinavir, nelfinavir, ritonavir, saquanivir
   b. Antiviral agents (general); lamivudine, foscamet, acyclovir, cidofovir, ganciclovir, valaciclovir
   c. amantadine, rimantadine, zanamivir, and oseltamivir

The present invention therefore provides a method of treating a viral condition comprising administering to a subject in need thereof a therapeutically effective amount of a compound of Formula (I) alone or in combination with a therapeutic agent selected from the group consisting of antibiotics, hormones, biologic response modifiers, analgesics, NSAIDs, DMARDs, glucocorticoids, immunosuppressants, immunomodulators, thrombolytic agents, antidepressants, gyrase inhibitors, beta lactam antibiotics, antifungal agents, and antiviral agents (as described above).

### EXAMPLES

The present invention may be further understood by reference to the following non-limiting examples. Examples of compounds of the present invention and procedures that may be used to prepare and identify useful compounds of the present invention are described below.

General Experimental

LC-MS data was obtained using gradient elution on a parallel MUX™ system, running four Waters 1525 binary HPLC pumps, equipped with a Mux-UV 2488 multichannel UV-Vis detector (recording at 215 and 254 nM) and a Leap Technologies HTS PAL Auto sampler using a Waters Xterra MS C18 4.6×50 mm column. A three minute gradient was run from 25% B (97.5% acetonitrile, 2.5% water, 0.05% TFA) and 75% A (97.5% water, 2.5% acetonitrile, 0.05% TFA) to 100% B. The system is interfaced with a Waters Micromass ZQ mass spectrometer using electrospray ionization. All MS data was obtained in the positive mode unless otherwise noted. ¹H NMR data was obtained on a Varian 400 MHz spectrometer.

Abbreviations used in the Examples are as follows:

BOC=tert-butoxycarbonyl
BOP=(1-benzotriazolyloxy)tris(dimethylamino)phosphonium hexafluorophosphate
d=day
DLAD=diisopropyl azodicarboxylate
DCC=dicyclohexylcarbodiimide
DCM=dichloromethane
DIEA=diisopropylethylamine
DMF=N,N-dimethylformamide
DMSO=dimethylsulfoxide
EDC=1-ethyl-3-(3-dimethylaminopropyl)-carbodiimide hydrochloride
ether=diethyl ether
EtOAc=ethyl acetate
FMOC=9-fluorenylmethoxycarbonyl
g=gram
h=hour
HBTU=O-benzotriazol-1-yl-N,N,N′,N′-tetramethyluronium hexafluorophosphate
HMPA=hexamethylphosphoric triamide
HOBt=1-hydroxybenzotriazole
Hz=hertz
L=liter
LAH=lithium aluminum hydride
LDA=lithium duisopropylamide
M=molar
m/z=mass to charge ratio
MeOH=methanol
mg=Milligram
min=minute
mL=milliliter
mM=millimolar
mmol=millimole
mol=mole
MS=mass spectrometry
N=normal
NMM=N-methylmorpholine, 4-methylmorpholine
NMR=nuclear magnetic resonance spectroscopy
PBS=phosphate buffered saline solution
ppm=parts per million
psi=pounds per square inch
R_{f}=relative TLC mobility
rt=room temperature
s.c.=subcutaneous
TEA=triethylamine
TFA=trifluoroacetic acid
THF=tetrahydrofuran
THP=tetrahydropyranyl

### Example 1

### N-[(1R)-1-(4-fluorophenyl)ethyl]-3-(2-isoquinoline-3-yl-1H-imidazol-4-yl)-5-isobutyrylamino)benzamide

### Step A; 3-chlorocarbonyl-5-nitro-benzoic acid methyl ester

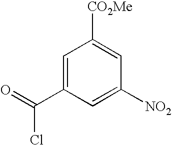

Thionyl chloride (30 mL) was added to 5-nitro-isophthalic acid monomethyl ester (4.5 g, 20 mmol). The reaction mixture was refluxed for 60 min, after cooling to rt, the thionyl chloride was removed in vacuo to afford 3-chlorocarbonyl-5-nitro-benzoic acid methyl ester, which was used directly in the next step.

¹H NMR (CDCl₃, 400 MHz): δ 4.08 (s, 3H), 9.06 (dd, 1H), 9.10 (dd, 1H), 9.14 (dd, 1H) ppm.

### Step B; 2-(3-methoxycarbonyl-5-nitro-benzoyl)-malonic acid diethyl ester

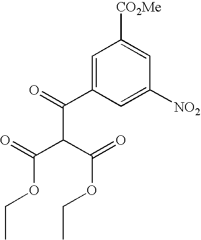

Diethyl malonate (4 g, 25 mmol) was added to a suspension of magnesium ethoxide (3.2 g, 28 mol) in dry THF (30 mL). The mixture was refluxed under nitrogen for 1.5 h. After cooling to rt, the mixture of magnesium diethyl malonate was treated with a solution of the above 3-chlorocarbonyl-5-nitro-benzoic acid methyl ester (1.1 equiv) in dry THF (25 mL). The resulted mixture was refluxed for 3 h. After removal of THF under vacuum, the residues were extracted with EtOAc (200 mL) and washed with 10% HCl aq. solution (50 mL). The organic phase was dried and concentrated in vacuo to give the crude 2-(3-methoxycarbonyl-5-nitro-benzoyl)-malonic acid diethyl ester.

¹H NMR (CDCl₃, 400 MHz): δ 1.28 (t, 6H), 3.38 (s, 1H), 4.06 (s, 3H), 4.20 (q, 4H), 9.10 (m, 3H) ppm.

### Step C; 3-acetyl-5-nitro-benzoic acid

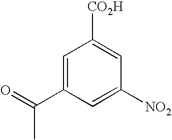

To a solution of the above crude 2-(3-methoxycarbonyl-5-nitro-benzoyl)-malonic acid diethyl ester in AcOH (12 mL) was added H₂ O(8 mL) and H₂SO₄ (1.5 mL). The reaction mixture was refluxed for 5 h. After cooling to rt, the reaction mixture was diluted with ethyl ether (250 mL) and washed with water (50 ml). The solvent was removed under reduced pressure to yield the crude 3-acetyl-5-nitro-benzoic acid.

¹H NMR (CDCl₃, 400 MHz): δ 2.78 (s, 3H), 8.91 (dd, 1H), 8.96 (dd, 1H), 9.04 (dd, 1H) ppm.

### Step D; 3-acetyl-5-nitro-benzoic acid methyl ester

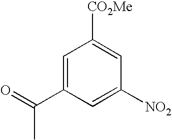

To a solution of the above crude 3-acetyl-5-nitro-benzoic acid in methanol (50 ml) was added 4M HCl in 1,4-dioxane (10 ml). The mixture was refluxed for 2 h. After removal of the organic solvent in vacuo, the residue was purified by flash column chromatography (hexanes then hexanes/EtOAc=1:1) to give 3-acetyl-5-nitro-benzoic acid methyl ester (Fw 223, 2.2 g, ca. 10 mmol, 50% yield over 4-Step). ¹H NMR (CDCl₃, 400 MHz): δ 2.74 (s, 3H), 4.03 (s, 3H), 8.89 (dd, 1H), 8.95 (dd, 1H), 9.05 (dd, 1H) ppm.

### Step E; 3-acetyl-5-amino-benzoic acid methyl ester

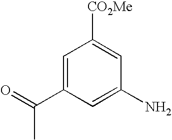

To a solution of 3-acetyl-5-nitro-benzoic acid methyl ester (Fw 223, 2.2 g, ca. 10 mmol) in AcOH (20 mL) was added iron powder (1 g). The mixture was refluxed for 20 min. After cooling to rt, the reaction mixture was diluted with EtOAc (150 mL) and methanol (50 mL). Then the suspension was filtered through a pad of filter aid. After removal of the organic solvent, the residue was extracted with EtOAc (150 mL) and washed with 5% aq. NaHCO₃ solution (50 mL). The organic phase was dried over MgSO4 and concentrated in vacuum to give 3-acetyl-5-amino-benzoic acid methyl ester.

### Step F; 3-Acetyl-5-isobutyrylamino-benzoic acid methyl ester

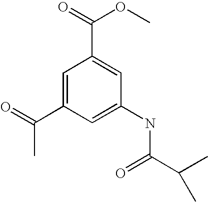

To a solution of the above aniline (10 mmol) in DCE (20 mL) was added pyridine (5 mL), the mixture was cooled to 0° C. then isobutyryl chloride (ca. 15 mol ) was added dropwise. The mixture was stirred at 0° C.-rt for 30 min-2 hr, then quenched by 5% aq. NH₄Cl solution (50 mL), and extracted with EtOAc (250 mL). The organic phase was dried over MgSO4 and the mixture was concentrated in vacuo. The residue was purified by flash column chromatography (hexanes then hexanes/EtOAc=1:2) to give 3-acetyl-5-isobutyrylamino-benzoic acid methyl ester.

### Step G; N-[3-(2-Bromo-acetyl)-5-propionyl-phenyl]isobutyramide

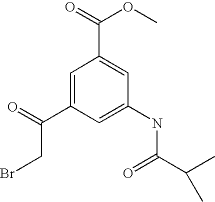

To a solution of the above 3-acetyl-5-isobutyrylamino-benzoic acid methyl ester (ca. 10 mmol) in THF (50 mL) was added pyrrolidone hydrotribromide (5 g, 10 mmol). The solution was refluxed for 30 min. The solution was allowed to cool to rt and 5% NaHCO₃ (50 mL) was added then the mixture was extracted with EtOAc (150 mL) and dried (MgSO₄). The solvent was removed in vacuo to afford N-[3-(2-bromo-acetyl)-5-propionyl-phenyl]-isobutyramide which was used directly in the next step.

### Step H; Isoquinoline-3-carboxylic acid 2-(3-isobutyrylamino-5-methoxycarbonyl-phenyl)-2-oxo-ethyl ester

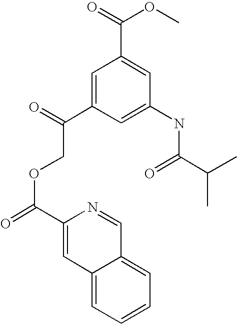

To a solution of N-[3-(2-bromo-acetyl)-5-propionyl-phenyl]-isobutyramide (Fw 395, 4 g, 10 mmol) in dry DMF (25 mL) was added of isoquinoline-3-carboxylic acid monohydrate (2.5 g, 13 mmol) and DIEA (5 mL) subsequently. The reaction mixture was stirred at rt for 1 h and then diluted with 150 mL of EtOAc, the organic phase was washed with 5% NaHCO₃ aq., dried over Na₂SO₄ and concentrated to afford the keto-ester was used directly in the next step.

### Step I; 3-Isobutyrylamino-5-(2-isoquinolin-3-yl-1H-imidazol-4-yl)-benzoic acid methyl ester

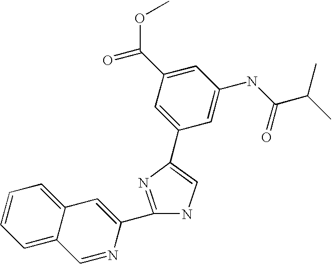

To a solution of the above keto-ester (ca. 10 mmol) in AcOH (30 mL) was added ammonium acetate (10 g); the reaction mixture was stirred at 135-150° C. for 2 h. After removal of AcOH under vacuum, the reaction mixtures were diluted with EtOAc (200 mL), and washed with 5% NaHCO₃ aq. solution, dried over MgSO₄ and concentrated under vacuum. The residues were purified by flash column chromatography (DCM then DCM/EtOAc=10:1 to 0:1) to give 3-Isobutyrylamino-5-(2-isoquinolin-3-yl-1H-imidazol-4-yl)-benzoic acid methyl ester.

### Step J; 3-Isobutyrylamino-5-(2-isoquinolin-3-yl-1H-imidazol-4-yl)-benzoic acid

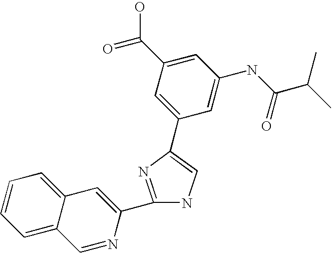

To a solution of the 3-Isobutyrylamino-5-(2-isoquinolin-3-yl-1H-imidazol-4-yl)-benzoic acid methyl ester in THF/methanol mixture (3:1) was treated with 4N LiOH (1-2 eq). The reaction mixture was stirred for 4 hr at RT and concentrated to remove the organic solvents. The aqueous solution was acidified with 1 N HCl (pH=3) and the solid (3-isobutyrylamino-5-(2-isoquinolin-3-yl-1H-imidazol-4-yl)-benzoic acid) was collected by filtration.

### Step K; N-[1-(R)-4-Fluoro-phenyl)-ethyl]-3-isobutyrylamino-5-(2-isoquinolin-3-yl-1H-imidazol-4-yl)-benzamide

To a stirred mixture of 3-isobutyrylamino-5-(2-isoquinolin-3-yl-1H-imidazol-4-yl)-benzoic acid (411 mg, 3.88 mmol) and HBTU (2.94 g, 7.77 mmol) in THF (10 mL) was added (R)-4-fluorophenethyl amine (500 mg, 2.59 mmol) followed by DIEA (2.8 mL, 15.54 mmol). The reaction mixture was stirred for 18 h and diluted with EtOAc and washed with water, 1N HCl, 5% NaHCO₃ solution and brine. The organic layer was dried over Na₂SO4 and evaporated. The crude obtained was purified on a silica gel column to afford the pale yellow crystalline product (0.67 g,), Example 1.

¹H NMR (CDCl₃): δ 9.1 (1H), 8.5 (1H), 8.2 (1H), 8.0 (1H), 7.9 (2H), 7.85 (1H), 7.65 (1H), 7.6 (2H), 7.5 (1H), 7.35 (2H), 7.0 (2H), 6.7 (1H), 5.3 (1H), 2.5 (1H), 1.6 (3H), 1.2 (6H) ppm. LC/MS: m/z 522 (M+1)⁺

The following Examples 2-4 were synthesized using appropriate reagents and using methods analogous to those described for Example 1, Steps A-K.

### Example 2

### N-[(1S)-1-(4-fluorophenyl)ethyl]-3-(2-isoquinoline-3-yl-1H-imidazol-4-yl)-5-isobutyrylamino-benzamide

¹H NMR (CDCl₃): δ 8.95 (1H), 8.45 (1H), 8.3 (1H), 8.2 (1H), 7.9 (1H), 7.8 (1H), 7.65 (1H), 7.6 (1H), 7.5 (1H), 7.4 (1H), 7.3 (2H), 6.9 (2H), 5.25 (1H), 2.5 (1H), 1.5 (3H), 1.2 (6H) ppm.

### Example 3

### N-[4-fluorophenethyl]-3-(2-isoquinoline-3-yl-1H-imidazol-4-yl)-5-isobutyrylamino-benzamide

¹H NMR (CDCl₃): δ 9.75 (1H), 8.5 (1H), 7.9 (5H), 7.7 (1H), 7.55 (1H), 7.45 (1H), 7.2 (2H), 7.0 (2H), 6.7 (1H), 3.65 (2H), 2.9 (2H), 2.5 (1H), 1.2 (6H) ppm.

### Example 4

### N-(4-Fluorobenzyl)-N-methyl-3-(2-isoquinoline-3-yl-1H-imidazol-4-yl)-5-isobutyrylamino-benzamide

¹H NMR (CDCl₃): δ 11.0 (1H), 9.0 (1H), 8.5 (1H), 8.2 (1H), 7.8 (2H), 7.7 (3H), 7.5 (1H), 7.3 (2H), 7.1 (1H), 7.0 (2H), 4.7 (2H), 4.5 (1H), 2.9 (3H), 2.5 (1H), 1.2 (6H) ppm.

### Example 5

### N-[(1R)-1-(4-fluorophenyl)ethyl]-3-(2-isoquinoline-3-yl-1H-imidazol-4-yl)-5-chlorobenzamide

To a solution of 3-acetyl-5-amino-benzoic acid methyl ester (1 g, 5 mmol) in HCl/AcOH (1.4 mL/2.0 mL) at −10° C. was added NaNO₂ and stirred for 30 min. CuCl and NaCl (excess) was added followed by AcOH (2 mL) at 0° C.-10° C. and stirred for 1 hr. The reaction mixture was diluted with EtOAc and water. The organic layer was suspended and washed with water, NaHCO₃ solution and brine. The crude product thus obtained was purified on a silica gel column to afford 3-acetyl-5-chloro-benzoic acid methyl ester (690 mg).

Using the methyl ester obtained above, the compound of Example 5 was employing procedures analogous to those described in Example 1, Steps G-K.

LC/MS: m/z 471 (M+1)⁺

### Example 6

### N-[(1R)-1-(4-fluorophenyl)ethyl]-3-(2-isoquinoline-3-yl-1H-imidazol-4-yl)benzamide

The compound of Example 6 was synthesized employing procedures analogous to those described in Example 1, Steps G-K.

¹H NMR (CDCl₃): δ 10.5 (1H), 9.2 (1H), 8.6 (1H), 8.3 (1H), 8.0 (3H), 7.75 (2H), 7.6 (1H), 7.5 (2H), 7.4 (2H), 7.2 (2H), 6.5 (1H), 5.35 (1H), 1.6 (3H) ppm.

### Example 7

### N-[(1S)-1-(4-fluorophenyl)ethyl]-3-(2-isoquinoline-3-yl-1H-imidazol-4-yl)benzamide

The title compound was synthesized from methyl-3-acetylbenzoate and other appropriate reagents, employing the procedures described in Example 1, Steps G-K.

¹H NMR (CDCl₃): δ 9.05 (1H), 8.7 (1 H), 7.9 (1 H), 7.8 (1H), 7.7 (2H), 7.5 (2H), 7.41 (1H), 7.25 (3H), 7.0 (4H), 5.7 (1H), 1.41 (3H) ppm. LC/MS: m/z 437 (M+1)⁺

Examples 8-11 were synthesized from methyl-3-acetylbenzoate and other appropriate reagents, employing the procedures described in Example 1, Steps G-K.

### Example 8

### N-[(1R)-1-phenylethyl]-3-(2-isoquinoline-3-yl-1H-imidazol-4-yl-benzamide

¹H NMR (CDCl₃): δ 9.3 (1H), 8.6 (1H), 8.25 (1H), 7.97 (1H), 7.9 (1H), 7.71 (1H), 7.61 (1H), 7.5 (1H), 7.45 (1H), 7.3 (7H), 5.14 (1H), 1.41 (3H) ppm. LC/MS: m/z 419 (M+1)⁺

### Example 9

### N-[1-(4-fluorophenyl)-2-methylpropyl]-3-(2-isoquinoline-3-yl-1 H-imidazol-4-yl-benzamide

¹H NMR (CDCl₃): δ 9.18 (1H), 8.6 (1H), 8.3 (1H), 7.95 (2H), 7.7 (1H), 7.6 (1H), 7.41 (1H), 7.3 (2H), 7.2 (2H), 7.0 (3H), 6.7 (1H), 4.9 (1H), 4.7 (1H), 0.95 (3H), 0.81 (3H) ppm. LC/MS: m/z 465 (M+1)⁺

### Example 10

### N-(2-isopropoxy-4-fluorophenyl)-3-(2-isoquinoline-3-yl-1H-imidazol-4-yl)-benzamide

¹H NMR (CDCl₃): δ 9.2 (1H), 8.63 (2H), 8.53 (1H), 8.4 (1 H), 8.05 (1H), 7.99 (1H), 7.91 (1H), 7.7 (2H), 7.6 (1H), 7.5 (2H), 6.67 (2H), 4.63 (1 H), 1.4 (6H) ppm. LC/MS: m/z 467 (M+1)⁺

### Example 11

### N-(2,4-dimethyl-4-fluorophenyl)-3-(2-isoquinoline-3-yl-1H-imidazol-4-yl)-benzamide

¹H NMR (MeOD): δ 9.4 (1H), 8.67 (1H), 8.53 (1H), 8.24 (1H), 6.1 (4H), 7.94 (1H), 7.8 (1H), 7.75 (1H), 6.92 (2H), 2.3 (6H) ppm. LC/MS: m/z 437 (M+1)⁺

### Example 12

### N-[(1R)-1-(4-fluorophenyl)ethyl]-2-isobutoxy-5-(2-isoquinoline-3-yl-1H-imidazol-4-yl)-benzamide

To a solution of ethyl 2-hydroxy-5-acetylbenzoate (2 g, 0.01 mol) in DMF were added cesium carbonate (3.9 g, 0.012 mol) and isobutyl bromide (4 mL, 0.012 mol). The reaction mixture was stirred at 50° C. for 16 h. The reaction mixture was cooled and partitioned between EtOAc and water. The organic layer was washed with water, brine and dried. The crude obtained after removal of the solvent was purified on a silica gel column to afford 5-acetyl-2-isobutoxy-benzoic acid ethyl ester, 2.5 g.

Using the ethyl ester obtained above, Example 12 was obtained using procedures described in Example 1, Steps G-K.

¹H NMR (CDCl₃): δ 10.5 (1H), 9.18 (1H), 8.6 (1H), 8.5 (1H), 8.4 (1H), 8.15 (1H), 7.97 (1H), 7.9 (1H), 7.7 (1H), 7.6 (1H), 7.45 (1H), 7.4 (2H), 7.0 (3H), 5.35 (1H), 3.9 (2H), 2.1 (1H), 1.6 (3H), 1.0 (6H) ppm. LC/MS: m/z 509 (M+1)⁺

### Example 13

### N-[(1R)-1-(4-fluorophenyl)ethyl]-3-(2-isoquinoline-3-yl-1H-imidazol-4-yl)-benzenesulfonamide

### Step A; 3-Acetyl-N-[(R)-1-(4-fluoro-phenyl)-ethyl]-benzenesulfonamide

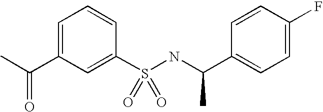

3-Acetyl-N-[(R)-1-(4-fluoro-phenyl)-ethyl]-benzenesulfonamide was synthesized by treatment of 3-acetylbenzenesulfonyl chloride in pyridine at 0° C. with (R)-1-(4-fluoro)phenylethylamine. Extractive workup with ethyl acetate afforded the desired product.

The compound of Example 13 was synthesized from 3-acetyl-N-[(R)-1-(4-fluoro-phenyl)-ethyl]-benzenesulfonamide employing analogous reagents and procedures to those described in Example 1, Steps J-I.

¹H NMR (CDCl₃): δ 10.65 (1H), 9.2 (1H), 8.6 (1H), 8.25 (1H), 8.05 (1H), 8.0 (2H), 7.7 (1H), 7.6 (2H), 7.4 (2H), 7.05 (2H), 6.8 (2H), 4.9 (1H), 4.5 (1H), 1.4 (3H) ppm. LC/MS: m/z 473 (M+1)⁺

### Example 14

### N-[(1R)-1-(4-fluorophenyl)ethyl]-3-(2-isoquinoline-3-yl-1H-imidazol-4-yl)-5-isobutylsulfonylamino-benzamide

### Step A; 3-Acetyl-5-(propane-2-sulfonylamino)-benzoic acid methyl ester

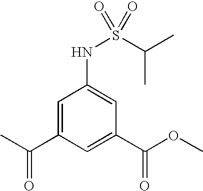

3-Acetyl-5-(propane-2-sulfonylamino)-benzoic acid methyl ester was synthesized by treatment of 3-acetyl-5-amino-benzoic acid methyl ester with isopropyl sulfonyl chloride in pyridine at 0° C., followed by extractive workup with ethyl acetate.

The compound of Example 14 was synthesized employing analogous reagents and procedures to those described in Example 1, Steps G-K.

¹H NMR (CDCl₃): δ 11.6 (1H), 9.0 (1H), 8.44 (1H), 8.1 (1H), 7.93 (1H), 7.8 (2H), 7,69 (1H), 7.58 (1H), 7.49 (1H), 7.38 (3H), 6.9 (2H), 5.4 (1H), 3.2 (1H), 1.5 (3H), 1.22 (6H) ppm.

### Examples 15

### N-[(1R)-1-(4-fluorophenyl)ethyl]-3-(2-isoquinoline-3-yl-1H-imidazol-4-yl)-5-pivaloylamino)benzamide

Example 15 was synthesized starting from the intermediate methyl 3-amino-5-acetylbenzoate, employing the following steps;

Step A; methyl ester hydrolysis analogous to Example 1, step J; and coupling with (R)-4-fluoro-alpha-phenethylamine analogous to Example 1, step K.

Step B; ketone bromination, analogous to analogous to Example 1, step G;

Step C; acyloxymethylene ketone formation, analogous to Example 1, step H;

Step D; imidazole formation, analogous to Example 1, step I.

¹H NMR (CDCl₃): δ 10.85 (1H), 9.15 (1H), 8.52 (1H), 8.2 (1H), 8.05 (1H), 7.95 (2H), 7.85 (1H), 7.7 (1H), 7.62 (1H), 7.6 (1H), 7.5 (1H), 7.35 (2H), 7.0 (2H), 6.88 (1H), 5.35 (1H), 1.62 (9H), 1.6 (3H) ppm.
Examples 16-20 were synthesized employing the appropriate reagents and procedures described for Example 15.

### Example 16

### N-[(1R)-1-(4-fluorophenyl)ethyl]-3-(2-isoquinoline-3-yl-1H-imidazol-4-yl)-5-(pentane-2-carbonylamino)benzamide

¹H NMR (CDCl₃): δ 9.0 (1H), 8.5 (1H), 8.2 (1H), 8.0 (3H), 7.9 (1H), 7.81 (1H), 7.65 (1H), 7.56 (1H), 7.4 (1H), 7.35 (2H), 7.05 (1H), 7.0 (2H), 5.3 (1H), 2.1 (1H), 1.7 (2H), 1.5 (2H), 1.6 (3H), 0.92 (6H) ppm.

### Example 17

### N-[(1R)-1-(4-fluorophenyl)ethyl]-3-(2-isoquinoline-3-yl-1H-imidazol-4-yl)-5-(2-fluoroisobutyrylamino)benzamide

¹H NMR (CDCl₃): δ 8.99 (1H), 8.4 (2H), 8.25 (1H), 8.0 (1H), 7.8 (2H), 7.6 (1H), 7.58 (1H), 7.48 (1H), 7.37 (2H), 6.9 (2H), 5.3 (1H), 3.68 (2H), 1.68 (3H), 1.62 (3H), 1.56 (3H) ppm.

### Example 18

### N-[(1R)-1-(4-fluorophenyl)ethyl]-3-((4-chlorophenyl)-1H-imidazol-4-yl)-5-isobutyrylamino-benzamide

¹H NMR (CDCl₃): δ 7.95 (1H), 7.8 (2H), 7.65 (3H), 7.4 (1H), 7.3 (2H), 7.1 (1H), 6.95 (2H), 5.25 (1H), 2.6 (1H), 1.5 (3H), 1.2 (6H) ppm.

### Example 19

### N-[(1R)-1-(4-fluorophenyl)ethyl]-3-((2-Naphthyl)-1H-imidazol-4-yl)-5-isobutyrylamino-benzamide

¹H NMR (CDCl₃): δ 8.34 (1H), 8.15 (1H), 8.0 (2H), 7.85 (3H), 7.72 (1H), 7.5 (3H), 7.42 (1H), 7.35 (2H), 7.0 (2H), 6.82 (1H), 5.3 (1H), 2.6 (1H), 1.6 (3H), 1.25 (6H) ppm.

### Example 20

### N-[(1R)-1-(4-fluorophenyl)ethyl]-3-((2-pyridyl)-1H-imidazol-4-yl)-5-isobutyrylamino-benzamide

¹H NMR (CDCl₃): δ 9.0 (1H), 8.4 (1H), 8.1 (1H), 8.0 (1H), 7.95 (2H), 7.3 (2H), 7.2 (2H), 7.1 (1H), 6.9 (2H), 5.25 (1H), 2.5 (1H), 1.55 (3H), 1.2 (6H) ppm.

### Example 21

### N-[1-(R)-(4-Fluoro-phenyl)-ethyl]-3-(isobutyryl-methyl-amino)-5-(2-isoquinolin-3-yl-1H-imidazol-4-yl)-benzamide

Methyl 3-acetyl-5-(isobutyrylamino)benzoate (500 mg, 1.9 mmol) in 5 mL of DMF was treated with NaH (91 mg, 2.28 mmol). The reaction mixture was stirred for 20 min at rt and then treated with MeI (0.5 mL, excess). After 2 h, the reaction mixture was neutralized with AcOH and partitioned between EtOAc and water. The organic layer was washed with water, NaHCO3 solution and brine. The crude obtained after removal of the solvent was purified on a silica gel column to afford 180 mg of methyl 3-acetyl-5-[isobutyryl(methyl)amino]benzoate.

The compound of Example 21 was synthesized employing the above intermediate methyl 3-acetyl-5-[isobutyryl(methyl)amino]benzoate, utilizing the appropriate reagents and procedures described as for Example 15, steps A-D.

¹H NMR (CDCl₃): δ 10.6 (1H), 9.2 (1H), 8.6 (1H), 8.23 (1H), 8.0 (1H), 7.9 (1H), 7.85 (1H), 7.75 (1H), 7.65 (1H), 7.6 (1H), 7.55 (1H), 7.4 (2H), 7.1 (2H), 6.65 (1H), 5.25 (1H), 3.3 (3H), 2.6 (1H), 1.6 (3H), 1.05 (6H) ppm.

### Example 22

### 3-{4-[3-(4-(4-fluorophenyl)-1H-imidazol-2-yl)phenyl]-1H-imidazol-2-yl}isoquinoline

4-Fluorophenacyl 3-(2-isoquinolin-3-y-1H-imidazol-4-yl)benzoate was synthesized from the intermediate 3-(2-isoquinolin-3-yl-1H-imidazol-4-yl)benzoic acid by treatment with 4-fluorophenacyl bromide as described in Example 1, Step H.

Example 22 was synthesized by treatment of 4-fluorophenacyl 3-(2-isoquinolin-3-yl-1H-imidazol-4-yl)benzoate according to the procedure for imidazole formation described in Example 1, Step I.

¹H NMR (MeOD): δ 9.4 (1H), 8.6 (1H), 8.53 (1H), 8.15 (1H), 8.05 (2H), 7.85 (4H), 7.7 (4H), 7.22 (2H) ppm. LC/MS: m/z 432 (M+1)⁺

### Example 23

### 3-[4-(3-{4-[(1R)-1-phenylethyl]-1H-imidazol-2-yl}phenyl)-1H-imidazol-2-yl]isoquinoline

To a solution of R-methyl phenylacetic acid (500 mg, 3.33 mmol) in THF at −10° C. was added NMM (0.362 mL, 3.3 mmol) and stirred for 15 min. Isobutyryl chloroformate (0.43 mL, 3.3 mmol) was added and stirring was continued for another 15 min. A freshly made solution of diazomethane (from 1-methyl-3-nitro-1 nitrosoguanidine and 20% NaOH in ether at 0° C) in ether was added at once to the reaction mixture at −10° C. and then warmed to rt. The reaction mixture was partitioned between EtOAc and water. The organic layer was washed with water and brine. The crude obtained after removal of the solvent was purified on a silica gel column to afford the diazoketone (500 mg). The diazoketone in ether at 0° C. was treated with 230 mg of conc. HBr and stirred for 1 hr at the same temperature. The reaction mixture partitioned between EtOAc and water. The EtOAc layer was washed with water and brine. The crude 1-bromo-3-(4-fluoro-phenyl)-butan-2-one obtained after removal of the solvent was used directly into the next reaction.

The compound of Example 23 was synthesized employing 1-bromo-3-(4-fluoro-phenyl)-butan-2-one and 3-(2-isoquinolin-3-yl-1H-imidazol-4-yl)benzoic acid according to the procedure described in Example 1, Steps H and I.

¹H NMR (CDCl₃): δ 9.2 (1H), 8.63 (1H), 8.45 (1H), 7.95 (3H), 7.74 (2H), 7.6 (2H), 7.48 (2H), 7.35 (5H), 4.1 (1H), 1.61 (3H) ppm. LC/MS: m/z 442 (M+1)⁺

### Example 24

### N-[(1R)-1-(4-fluorophenyl)ethyl]-3-(2-isoquinoline-3-yl-1H-imidazol-4-yl)-5-phenylbenzamide

Methyl 3-bromo-5-acetylbenzoate was treated with phenylboronic acid and tetrakis(triphenylphosphine)palladium in aqueous sodium carbonate under microwave irradiation to afford, after neutralization and extractive workup, 3-phenyl-5-acetylbenzoic acid.

3-Phenyl-5-acetylbenzoic acid was processed using appropriate reagents and utilizing the procedures noted for Example 1, steps K, G, H, and I, in turn, to afford the compound of Example 24. LC/MS: m/z 513 (M+1)⁺

### Example 25

### N-[(1R)-1-(4-fluorophenyl)ethyl]-3-(2-isoquinoline-3-yl-1H-imidazol-4-yl)-5-(2-fluoroisobutyryl-methylamino)benzamide

3-Acetyl-5-aminobenzamide was treated with 2-fluoroisobutyric acid and HBTU according to the procedure in Example 1, step K to afford the anilide.

3-Acetyl-5-(2-fluoroisobutyrylamino)-benzamide was alkylated with methyl iodide according to the procedure of Example 12, step A, to afford the methylamide.

3-Acetyl-5-(2-fluoroisobutyryl-methylamino)-benzamide was processed employing appropriate reagents and following the procedures of Example 1 steps J, K, G, H, and I, in turn, to afford the compound of Example 25. LC/MS: m/z 554 (M+1)⁺

### Example 26

### N-[(1R)-(4-Fluoro-phenyl)-ethyl]-3-(2-isoquinolin-3-yl-3-methyl-3H-imidazol-4-yl)-benzamide

3-(2-Isoquinolin-3-yl-3H-imidazol-4-yl)-benzoic acid methyl ester was synthesized from methyl-3-acetylbenzoate and other appropriate reagents, employing the procedures described in Example 1, Steps G-I.

3-(2-Isoquinolin-3-yl-3H-imidazol-4-yl)-benzoic acid methyl ester was dissolved in DMF and treated with excess methyl iodide and excess DIEA. The reaction was heated until TLC analysis showed consumption of the starting material. The reaction was diluted with EtOAc, quenched with water and the organic portion was separated. After drying over Na₂SO₄, the volatiles were removed in vacuo. The crude oil was purified using silica gel chromatography to afford 3-(2-isoquinolin-3-yl-1-methyl-1H-imidazol-4-yl)-benzoic acid methyl ester.

The compound of Example 26 was synthesized from 3-(2-isoquinolin-3-yl-1-methyl-1H-imidazol-4-yl)-benzoic acid methyl ester using the appropriate reagents and procedures as described in Example 1, Steps J and K. LC/MS: m/z 452 (M+1)⁺

### Example 27

### 3-[2-(6,7-Dimethoxy-isoquinolin-3-yl)-3H-imidazol-4-yl]-5-(2-fluoro-2-methyl-propionylamino)-N-[(1R)-(4-fluoro-phenyl)-ethyl]-benzamide

3-Acetyl-5-nitro-benzoic acid methyl ester was manipulated using the procedures described in Example 1, Steps J, K, E, K, and G to provide 3-(2-bromo-acetyl)-5-(2-fluoro-2-methyl-propionylamino)-N-[(1R)-(4-fluoro-phenyl)-ethyl]-benzamide.

Example 27 was synthesized from 3-(2-bromo-acetyl)-5-(2-fluoro-2-methyl-propionylamino)-N-[(1R)-(4-fluoro-phenyl)-ethyl]-benzamide and 6,7-dimethoxy-isoquinoline-3-carboxylic acid following the procedures described in Example 1, Steps H and I. LC/MS: m/z 601 (M+1)⁺

### Example 28

### N,N′-Bis-[(1R)-(4-fluoro-phenyl)-ethyl]-5-(2-isoquinolin-3-yl-3H-imidazol-4-yl)-isophthalamide

### Example 29

### N-[(1R)-(4-Fluoro-phenyl)-ethyl]-5-(2-isoquinolin-3-yl-3H-midazol-4-yl)-isophthalamic acid

5-Acetyl-isophthalic acid dimethyl ester was employed with appropriate reagents and procedures as described in Example 1, Steps G-I to afford 5-(2-isoquinolin-3-yl-3H-midazol-4-yl)-isophthalic acid dimethyl ester.

5-(2-isoquinolin-3-yl-3H-midazol-4-yl)-isophthalic acid dimethyl ester (165 mg) was dissolved in 2 mL of dioxane, treated with 500 μL of 15% aqueous KOH, and heated at 75° C. overnight. The mixture was concentrated in vacuo, diluted with water, and acidified to pH 3 with 1 N HCl. The resulting solid was collected and dried in vacuo to afford 135 mg of 5-(2-isoquinolin-3-yl-3H-imidazol-4-yl)-isophthalic acid.

5-(2-Isoquinolin-3-yl-3H-imidazol-4-yl)-isophthalic acid (135 mg) was treated with HBTU (178 mg, 1.25 equiv.) and DIEA (165 μL, 2.5 equiv.) in DMF. After stirring at rt for 15 min, (1R)-(4-fluoro-phenyl)-ethylamine (52 mg, 1 equiv) was added and the reaction was stirred overnight. Cold water was added to the reaction mixture and the resulting solid was collected and dried in vacuo. Purification via silica gel chromatography using 1% MeOH, 20% EtOAc in DCM afforded 47 mg of the compound of Example 28. Further elution with 5% MeOH, 25% EtOAc and 0.5% HOAc in DCM afforded 52 mg of the compound of Example 29.
Example 28; LC/MS: m/z 603 (M+1)⁺
Example 29; LC/MS: m/z 482 (M+1)⁺

### Example 30

### N-[(1R)-(4-Fluoro-phenyl)-ethyl]-N′-isopropyl-5-(2-isoquinolin-3-yl-3H-imidazo-4-yl)-isophthalamide

N-[(1R)-(4-Fluoro-phenyl)-ethyl]-5-(2-isoquinolin-3-yl-3H-midazol-4-yl)-isophthalamic acid (Example 29) was dissolved in DMF, cooled to 0° C., treated with two equivalents of DIEA and 1.5 equivalents of fluoro-N,N,N′,N′-tetramethylformamidinium hexafluorophosphate (TFFH). After 20 min, three equivalents of isopropyl amine were added to the mixture and the resulting solution was stirred for 50 min. TLC analysis indicated consumption of the starting material. The reaction was diluted with EtOAc and quenched with sat. aq. bicarbonate. The organic layer was separated from the aqueous and dried with Na₂SO₄. After removal of the organic solvent with reduced pressure, the crude material was purified via silica gel chromatography to provide Example 30.

LC/MS: m/z 523 (M+l)⁺

### Example 31

### N-[(1R)-(4-Fluoro-phenyl)-ethyl]-3-(2-isoquinolin-3-yl-3H-imidazol-4-yl)-5-(2-methyl-propenyl)-benzamide

3-Acetyl-5-bromo-benzoic acid methyl ester was dissolved in DMF and treated with one equivalent of 2-methyl-propenyl tributyltin and tetrakis(triphenylphosphine)palladium (10 mol %). The reaction was subjected to microwave heating (150° C.) for 15 min. TLC analysis indicated high conversion of starting material to a new compound. The reaction was quenched with 10% aqueous KF, extracted with EtOAc and the layers were separated. After drying the organic portion over Na₂SO₄, the solvent was removed in vacuo. Purification by silica gel chromatography provided 3-acetyl-5-(2-methyl-propenyl)-benzoic acid methyl ester.

The compound of example 31 was synthesized from 3-acetyl-5-(2-methyl-propenyl)-benzoic acid methyl ester employing the procedures analogous to those described in Example 1, Steps G-K.

LC/MS: m/z 492 (M+1)⁺

### Example 32

### 3-(5-Dimethylamino-naphthalene-1-sulfonylamino)-N-[(R)-1-(4-fluoro-phenyl)-ethyl]-5-(2-isoquinolin-3-yl-3H-imidazol-4-yl)-benzamide

To 3-amino-N-[(R)-1-(4-fluoro-phenyl)-ethyl]-5-(2-isoquinolin-3-yl-3H-imidazol-4-yl)-benzamide (262 mg, 0.58 mmol) in 3 ml pyridine at rt was added dansyl chloride (135 mg, 0.5 mmol). The mixture was stirred at rt for 5 h, diluted with ice water, filtered and dried. The solid was precipitated from DCM-ether and washed twice with ether-hexane mixture to afford Example 32. LC-MS: 686 (M+2)⁺

### Example 33

### N-[1-(4-Fluoro-phenyl)-ethyl]-4-(2-isoquinolin-3-yl-3H-imidazol-4-yl)-benzamide

The compound of Example 33 was synthesized from 4-acetyl benzoic acid employing the procedures analogous to those described in Example 1, Steps K, G, H and I.

¹H NMR (CDCl₃): δ 9.19 (m, 2H), 8.12 (d, 1H), 8.00 (d, 1H), 7.89 (m, 5H), 7.81 (m, 1H), 7.70 (m, 1H), 7.42 (m, 2H), 7.05 (m, 2H), 5.30 (m, 1H), 1.62 (d, 3H) ppm. LC/MS: m/z 437 (M+1)⁺

### Example 34

### 2-Ethoxy-N-[(1R)-(4-fluoro-phenyl)-ethyl]-5-(2-isoquinolin-3-yl-3H-imidazol-4-yl)-benzamide

5-Acetyl-2-hydroxy-benzoic acid was dissolved in DMF and heated in the presence of excess iodoethane and excess DIEA until TLC indicated consumption of the starting material. The reaction was diluted with EtOAc and quenched with water. After separation of the layers, the organic portion was dried over Na₂SO₄ and concentrated in vacuo. Purification by silica gel chromatography afforded 5-acetyl-2-ethoxy-benzoic acid ethyl ester.

The compound of Example 34 was synthesized using 5-acetyl-2-ethoxy-benzoic acid ethyl ester and the other appropriate reagents employing the procedures described in Example 1, Steps G-K.

¹H NMR (CDCl₃): δ 9.19 (s, 1H), 8.64 (s, 1H), 8.53 (d, 1H), 8.44 (d, 1H), 8.06 (m, 1H), 7.99 (d, 1H), 7.80 (m, 1H), 7.73 (m, 2H), 7.38 (m, 2H), 7.02 (m, 2H), 5.12 (m, 1H), 3.95 (q, 2H), 1.59 (t, 3H), 1.48 (d, 3H) ppm. LC/MS: m/z 482 (M+1)⁺

### Example 35

### N-[(1R)-(4-Fluoro-phenyl)-ethyl]-3-(2-isoquinolin-3-yl-3H-imidazol-4-yl)-5-(piperidine-1-carbonyl)-benzamide

Example 35 was synthesized using the compound of Example 31 and piperidine according to the procedure described in Example 1, Step K.

¹H NMR (CDCl₃): δ 9.22 (s, 1H), 8.63 (s, 1H), 8.53 (s, 1H), 8.32 (s, 1H), 8.00 (d, 1H), 7.96 (d, 1H), 7.74 (m, 1H), 7.64 (m, 2H), 7.42 (m, 2H), 7.07 (m, 2H), 6.56 (d, 1H), 5.39 (m, 1H), 1.66 (d, 3H), 1.58 (m, 4H), 1.26 (m, 4H), 0.86 (m, 2H) ppm. LC/MS: m/z 549 (M+1)⁺

### Example 36

### Morpholine-4-carboxylic acid [3-[(1R)-(4-fluoro-phenyl)-ethylcarbamoyl]-5-(2-isoquinolin-3-yl-3H-imidazol-4-yl)-phenyl]-amide

3-Amino-N-[(1R)-(4-fluoro-phenyl)-ethyl]-5-(2-isoquinolin-3-yl-3H-imidazol-4-yl)-benzamide was dissolved in THF and treated with benzotriazole-1-carbonyl chloride. Once consumption of starting material was confirmed by LCMS analysis, excess morpholine was added and the reaction was stirred overnight. The reaction was diluted with EtOAc and quenched with water. After separation of the layers,: the organic portion was dried over Na₂SO₄ and concentrated in vacuo. Purification by silica gel chromatography afforded Example 436.

¹H NMR (CDCl₃): δ 9.09 (m, 1H), 8.46 (s, 1H), 7.91 (m, 3H), 7.78 (m, 2H), 7.66 (m, 1H), 7.57 (m, 1H), 7.38 (m, 3H), 7.00 (m, 2H), 5.29 (m, 1H), 3.70 (s, 4H), 3.48 (s, 4H), 1.56 (d, 3H) ppm. LC/MS: m/z 566 (M+1)⁺

### Example 37

### 3-(2-Amino-2-methyl-propionylamino)-N-[(1R)-(4-fluoro-phenyl)-ethyl]-5-(2-isoquinolin-3-yl-3H-imidazol-4-yl)-benzamide

3-Amino-N-[(1R)-(4-fluoro-phenyl)-ethyl]-5-(2-isoquinolin-3-yl-3H-imidazol-4-yl)-benzamide was combined with N-FMOC-amino isobutyric acid according to the procedure described in Example 1, Step K. Removal of the FMOC protecting group was accomplished using excess piperidine in DCM. Purification by silica gel chromatography afforded Example 37.

¹H NMR (CDCl₃): δ 10.17 (s, 1H), 9.17 (s, 1H), 8.57 (s, 1H), 8.28 (s, 1H), 8.03 (s, 1H), 7.97 (d, 1H), 7.89 (d, 1H), 7.71 (m, 1H), 7.60 (m, 1H), 7.54 (s, 1H), 7.38 (m, 2H), 7.03 (m, 2H), 6.76 (d, 1H), 5.36 (m, 1H), 1.61 (d, 3H), 1.49 (s, 6H) ppm. LC/MS: m/z 538 (M+1)⁺

### Example 38

### Morpholine-2-carboxylic acid [3-[(1R)-(4-fluoro-phenyl)-ethylcarbamoyl]-5-(2-isoquinolin-3-yl-3H-imidazol-4-yl)-phenyl]-amide

3-Amino-N-[(1R)-(4-fluoro-phenyl)-ethyl]-5-(2-isoquinolin-3-yl-3H-imidazol-4-yl)-benzamide was combined with N-FMOC 2-carboxy morpholine according to the procedure described in Example 1, Step K. Removal of the FMOC protecting group was accomplished using excess piperidine in DCM. Purification by silica gel chromatography afforded Example 38.

¹H NMR (CDCl₃): δ 9.20 (s, 1H), 8.58 (d, 1H), 8.28 (s, 1H), 8.19 (d, 1H), 7.93 (d, 1H), 7.85 (d, 1H), 7.74 (m, 1H), 7.62 (m, 1H), 7.58 (s, 1H), 7.39 (m, 2H), 7.04 (m, 2H), 6.63 (m, 1H), 5.36 (m, 1H), 4.09 (m, 1H), 3.75 (m, 2H), 3.44 (d, 2H), 2.82 (m, 2H), 1.63 (d, 3H), ppm. LC/MS: m/z 566 (M+1)⁺

### Example 39

### 3-(2S-Amino-propionylamino)-N-[(1R)-(4-fluoro-phenyl)-ethyl]-5-(2-isoquinolin-3-yl-3H-imidazol-4-yl)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC L-alanine according to the procedure described in Example 1, Step K. The BOC protecting group was removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 39.

¹H NMR (CDCl₃): δ 9.26 (s, 1H), 9.07 (s, 1H), 8.43 (s, 1H), 8.20 (s, 1H), 8.10 (m, 2H), 8.04 (d, 1H), 7.91 (s, 1H), 7.83 (m, 1H), 7.76 (m, 1H), 7.48 (m, 2H), 7.02 (m, 2H), 5.27 (m, 1H), 4.18 (m, 1H), 1.65 (m, 6H) ppm. LC/MS: m/z 524 (M+1)⁺

### Example 40

### 3-(2R-Amino-propionylamino)-N-[(1R)-(4-fluoro-phenyl)-ethyl]-5-(2-isoquinolin-3-yl-3H-imidazol-4-yl)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC D-alanine according to the procedure described in Example 1, Step K. The BOC protecting group was removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 40.

¹H NMR (CDCl₃): δ 9.40 (s, 1H), 8.93 (s, 1H), 8.78 (s, 1H), 8.27 (s, 1H), 8.18 (m, 2H), 8.08 (m, 2H), 7.88 (m, 1H), 7.80 (m, 1H), 7.49 (m, 2H), 7.07 (m, 2H), 5.25 (m, 1H), 4.24 (m, 1H), 1.67 (d, 3H), 1.62 (d, 3H) ppm. LC/MS: m/z 524 (M+1)⁺

### Example 41

### Piperidine-4-carboxylic acid [3-[(1R)-(4-fluoro-phenyl)-ethylcarbamoyl]-5-(2-isoquinolin-3-yl-3H-imidazol-4-yl)-phenyl]-amide Dihydrochloride

3-Amino-N-[(1R)-(4-fluoro-phenyl)-ethyl]-5-(2-isoquinolin-3-yl-3H-imidazol-4-yl)-benzamide was combined with N-BOC isonipecotic acid according to the procedure described in Example 1, Step K. The BOC protecting group was removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 41.

¹H NMR (CD₃OD): δ 9.45 (s, 1H), 8.91 (s, 1H), 8.73 (s, 1H), 8.32 (s, 1H), 8.21 (d, 1H), 8.13 (m, 2H), 8.02 (s, 1H), 7.92 (m, 1H), 7.84 (m, 1H), 7.48 (m, 2H), 7.08 (m, 2H), 5.26 (m, 1H), 3.51 (d, 2H), 3.13 (m, 2H), 2.87 (m, 1H), 2.18 (m, 2H), 2.04 (m, 2H), 1.61 (d, 3H) ppm. LC/MS: m/z 564 (M+1)⁺

### Example 42

### Acetic acid 1-[3-[(1R)-(4-fluoro-phenyl)-ethylcarbamoyl]-5-(2-isoquinolin-3-yl-3H-imidazol-4-yl)-phenylcarbamoyl]-ethyl ester

3-Amino-N-[(1R)-(4-fluoro-phenyl)-ethyl]-5-(2-isoquinolin-3-yl-3H-imidazol-4-yl)-benzamide was combined with 2-acetoxy propionic acid according to the procedure described in Example 1, Step K. Purification by silica gel chromatography provided Example 42.

¹H NMR (CDCl₃): δ 9.21 (s, 1H), 8.50 (s, 1H), 8.09 (m, 1H), 8.02 (s, 1H), 7.99 (d, 1H), 7.96 (d, 1H), 7.82(s, 1H), 7.75 (m, 1H), 7.64 (m, 1H), 7.56 (s, 1H), 7.42 (m, 2H), 7.05 (m, 2H), 5.31 (m, 1H), 5.25 (q, 1H), 2.25 (s, 3H), 1.61 (m, 6H) ppm. LC/MS: m/z 567 (M+1)⁺

### Example 43

### N-[(1R)-(4-Fluoro-phenyl)-ethyl]-3-(2-hydroxy-propionylamino)-5-(2-isoquinolin-3-yl-3H-imidazol-4-yl)-benzamide

Acetic acid 1-[3-[(1 R)-(4-fluoro-phenyl)-ethylcarbamoyl]-5-(2-isoquinolin-3-yl-3H-imidazol-4-yl)-phenylcarbamoyl]-ethyl ester (Example 42) was dissolved in MeOH and treated with K₂CO₃ at rt overnight. After removal of the MeOH under reduced pressure, the crude product was dissolved in DCM and filtered. The filtrate was concentrated and triturated with ethyl ether/hexanes to provide Example 43.

¹H NMR (CDCl₃): δ 9.07 (s, 1H), 8.68 (s, 1H), 8.51 (s, 1H), 7.99 (m, 1H), 7.88 (m, 2H), 7.77 (m, 1H), 7.69 (m, 1H), 7.58 (m, 1H), 7.44 (s, 1H), 7.39 (m, 2H), 7.04 (m, 2H), 6.89 (m, 1H), 5.33 (m, 1H), 4.36 (m, 1H), 3.45 (m, 1H), 1.63 (d, 3H), 1.51 (d, 3H) ppm. LC/MS: m/z 524 (M+1)⁺

### Example 44

### 3-(2-Amino-acetylamino)-N-[(1R)-(4-fluoro-phenyl)-ethyl]-5-(2-isoquinolin-3-yl-3H-imidazol-4-yl)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC glycine according to the procedure described in Example 1, Step K. The BOC protecting group was removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 44.

¹H NMR (CD₃OD): δ 9.43 (s, 1H), 8.76 (s, 1H), 8.37 (s, 1H), 8.19 (m, 2H), 8.14 (s, 1H), 8.07 (m, 2H), 7.90 (m, 1H), 7.83 (m, 1H), 7.48 (m, 2H), 7.07 (m, 2H), 5.26 (m, 1H), 3.98 (s, 2H), 1.62 (d, 3H) ppm. LC/MS: m/z 510 (M+1)⁺

### Example 45

### 3-(3-Amino-propionylamino)-N-[(1R)-(4-fluoro-phenyl)-ethyl]-5-(2-isoquinolin-3-yl-3H-imidazol-4-yl)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC 3-amino propionic acid according to the procedure described in Example 1, Step K. The BOC protecting group was removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 45.

¹H NMR (CD₃OD): δ 9.25 (s, 1H), 8.42 (s, 1H), 8.11 (s, 1H), 8.03 (m, 2H), 7.92 (m, 2H), 7.74 (m, 2H), 7.60 (m, 2H), 7.44 (m, 2H), 7.06 (m, 2H), 5.23 (m, 1H), 3.43 (t, 2H), 2.59 (t, 2H), 1.61 (d, 3H) ppm. LC/MS: m/z 524 (M+1)⁺

### Example 41

### N-[(1R)-(4-Fluoro-phenyl)-ethyl]-3-(2-isoquinolin-3-yl-3H-imidazol-4-yl)-5-(2-methanesulfonylamino-acetylamino)-benzamide

The compound of Example 44 (3-(2-amino-acetylamino)-N-[(1R)-(4-fluoro-phenyl)-ethyl]-5-(2-isoquinolin-3-yl-3H-imidazol-4-yl)-benzamide) was dissolved in DCM and treated with two equivalents of K₂CO₃ as well as a catalytic amount of tetrabutylammonium bromide. Two equivalents of methanesulfonyl chloride were added and the reaction was heated to 45° C. for two h. LCMS analysis indicated complete consumption of starting material. The crude reaction mixture was concentrated onto silica gel without workup. Purification by silica gel chromatography afforded Example 46.

¹H NMR (CDCl₃): δ 9.19 (s, 1H), 8.55 (s, 1H), 8.08 (s, 1H), 8.04 (s, 1H), 7.99 (d, 1H), 7.96 (d, 1H), 7.82 (s, 1H), 7.77 (m, 1H), 7.65 (m, 1H), 7.56 (s, 1H), 7.43 (m, 2H), 7.05 (m, 2H), 5.33 (m, 1H), 4.00 (s, 2H), 3.41 (m, 1H), 3.07 (s, 3H), 1.63 (d, 3H) ppm. LC/MS: m/z 588 (M+1)⁺

### Example 47

### N-[(1R)-(4-Fluoro-phenyl)-ethyl]-3-guanidino-5-(2-isoquinolin-3-yl-3H-imidazol-4-yl)-benzamide Dihydrochloride

3-Amino-N-[(1R)-(4-fluoro-phenyl)-ethyl]-5-(2-isoquinolin-3-yl-3H-imidazol-4-yl)-benzamide was treated with bis-BOC-guanyl pyrazole and K₂CO₃ in dioxane under microwave heating (90° C., 30 min). The crude reaction mixture was concentrated onto silica gel. Purification by silica gel chromatography afforded the desired bis-BOC protected intermediate. The BOC protecting groups were removed by dissolving in DCM and addition of excess 4 N HCl in dioxane. After stirring for 1 h, the volatile components were removed under reduced pressure. The residue was triturated with ethyl ether and the precipitated solid was filtered and dried under vacuum to afford Example 47.

LC/MS: m/z 495 (M+1)⁺

### Example 48

### 3-(2-Acetylamino-acetylamino)-N-[(1R)-(4-fluoro-phenyl)-ethyl]-5-(2-isoquinolin-3-yl-3H-imidazol-4-yl)-benzamide

3-Amino-N-[(1R)-(4-fluoro-phenyl)-ethyl]-5-(2-isoquinolin-3-yl-3H-imidazol-4-yl)-benzamide was combined with N-acetyl glycine according to the procedure described in Example 1, Step K. Purification by silica gel chromatography afforded Example 48.

¹H NMR (CD₃OD): δ 9.31 (s, 1H), 8.49 (s, 1H), 8.16 (s, 1H), 8.09 (d, 1H), 8.05 (s, 1H), 8.00 (d, 1H), 7.91 (s, 1H), 7.79 (t, 1H), 7.68 (m, 1H), 7.64 (s, 1H), 7.46, (m, 2H), 7.07 (m, 2H), 5.25 (m, 1H), 4.05 (s, 2H), 2.06 (s, 3H), 1.59 (d, 3H) ppm. LC/MS: m/z 552 (M+1)⁺

### Example 49

### N-[(1R)-(4-Fluoro-phenyl)-ethyl]-3-(2-isoquinolin-3-yl-3H-imidazol-4-yl)-5-(2-methylamino-propionylamino)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC-N-methyl L-alanine according to the procedure described in Example 1, Step K. The BOC protecting group was removed by dissolving in DCM followed by addition of excess 4 N HCl in dioxane. After stirring for 1 h, the volatile components were removed under reduced pressure. The residue was triturated with ethyl ether and the precipitated solid was filtered and dried under vacuum to afford Example 49.

¹H NMR (CD₃OD): δ 9.41 (s, 1H), 8.72 (s, 1H), 8.29 (s, 1H), 8.16 (m, 2H), 8.08 (m, 2H), 7.96 (s, 1H), 7.90 (m, 1H), 7.81 (m, 1H), 7.47 (m, 2H), 7.07 (m, 2H), 5.26 (m, 1H), 4.11 (m, 1H), 2.78 (s, 3H), 1.68 (s, 3H), 1.62 (d, 3H), 1.37 (m, 1H) ppm. LC/MS: m/z 538 (M+1)⁺

### Example 50

### N-[(1R)-(4-Fluoro-phenyl)-ethyl]-3-(2-isoquinolin-3-yl-3H-imidazol-4-yl)-5-(2-methylamino-acetylamino)-benzamide Dihydrochloride

3-Amino-N-[(1R)-(4-fluoro-phenyl)-ethyl]-5-(2-isoquinolin-3-yl-3H-imidazol-4-yl)-benzamide was combined with N-BOC-N-methyl glycine according to the procedure described in Example 1, Step K. The BOC protecting group was removed by dissolving in DCM followed by addition of excess 4 N HCl in dioxane. After stirring for 1 h, the volatile components were removed under reduced pressure. The residue was triturated with ethyl ether and the precipitated solid was filtered and dried under vacuum to afford Example 50.

¹H NMR (CD₃OD): δ 9.34 (s, 1H), 8.89 (d, 1H), 8.66 (s, 1H), 8.23 (s, 1H), 8.12 (m, 2H), 8.03 (m, 1H), 7.83 (m, 2H), 7.75 (m, 1H), 7.47 (m, 2H), 7.08 (m, 2H), 5.27 (m, 1H), 4.08 (s, 2H), 2.84 (s, 3H), 1.62 (d, 3H) ppm. LC/MS: m/z 524 (M+1)⁺

### Example 51

### N-[3-[(1R)-(4-Fluoro-phenyl)-ethylcarbamoyl]-5-(2-isoquinolin-3-yl-3H-imidazol-4-yl)-phenyl]-succinamic acid methyl ester

3-Amino-N-[(1R)-(4-fluoro-phenyl)-ethyl]-5-(2-isoquinolin-3-yl-3H-imidazol-4-yl)-benzamide was combined with succinic acid mono-methyl ester according to the procedure described in Example 1, Step K. Purification by silica gel chromatography afforded Example 51.

¹H NMR (CDCl₃): δ 9.24 (s, 1H), 8.54 (s, 1H), 8.19 (d, 1H), 8.07 (s, 1H), 8.01 (m, 2H), 7.85 (s, 1H), 7.78 (m, 1H), 7.66 (m, 1H), 7.59 (s, 1H), 7.42 (m, 2H), 7.04 (m, 2H), 5.31 (m, 1H), 3.83 (s, 2H), 3.74 (s, 3H), 2.77 (s, 2H), 1.63 (d, 3H) ppm. LC/MS: m/z 567 (M+1)⁺

### Example 52

### N-[3-[(1R)-(4-Fluoro-phenyl)-ethylcarbamoyl]-5-(2-isoquinolin-3-yl-3H-imidazol-4-yl)-phenyl]-succinamide

3-Amino-N-[(1R)-(4-fluoro-phenyl)-ethyl]-5-(2-isoquinolin-3-yl-3H-imidazol-4-yl)-benzamide was combined with succinamic acid according to the procedure described in Example 1, Step K. Purification by silica gel chromatography afforded Example 52.

¹H NMR (CD₃OD): δ 9.30 (s, 1H), 8.79 (d, 1H), 8.48 (s, 1H), 8.14 (s, 1H), 8.09 (d, 1H), 8.02 (s, 1H), 7.99 (d, 1H), 7.89 (s, 1H), 7.79 (m, 1H), 7.67 (m, 1H), 7.64 (s, 1H), 7.45 (m, 2H), 7.08 (m, 2H), 5.26, (m, 1H), 2.74 (t, 2H), 2.64 (t, 2H), 1.59 (d, 3H) ppm. LC/MS: m/z 552 (M+1)⁺

### Example 53

### N-[3-[1-(4-Fluoro-phenyl)-ethylcarbamoyl]-5-(2-isoquinolin-3-yl-3H-imidazol-4-yl)-phenyl]-succinamic acid

N-[3-[(1R)-(4-Fluoro-phenyl)-ethylcarbamoyl]-5-(2-isoquinolin-3-yl-3H-imidazol-4-yl)-phenyl]-succinamic acid methyl ester (Example 51) was used with appropriate reagents and procedures as described in Example 1, Step J to provide Example 53.

LC/MS: m/z 553 (M+1)⁺

### Example 54

### N-[(1R)-(4-Fluoro-phenyl)-ethyl]-5-(2-isoquinolin-3-yl-3H-imidazol-4-yl)-isophthalamic acid methyl ester

N-[(1R)-(4-Fluoro-phenyl)-ethyl]-5-(2-isoquinolin-3-yl-3H-midazol-4-yl)-isophthalamic acid (Example 29) was dissolved in DMF and treated with excess methyl iodide and excess DIEA. The reaction was stirred for 6 h at rt at which time TLC analysis showed consumption of the starting material. The reaction was diluted with EtOAc, quenched with water and the organic layer was isolated. After drying over Na₂SO₄, the volatiles were removed in vacuo. The crude oil was purified using silica gel chromatography to afford Example 54.

¹H NMR (CDCl₃): δ 9.18 (s, 1H), 8.53 (s, 1H), 8.40 (s, 1H), 7.99 (m, 1H), 7.76 (m, 1H), 7.67 (m, 1H), 7.62 (s, 1H), 7.57 (m, 2H), 7.48 (m, 2H), 7.05 (m, 2H), 5.38 (m, 1H), 3.96 (s, 3H), 1.70 (d, 3H), ppm. LC/MS: m/z 495 (M+1)⁺

### Example 55

### Process for the synthesis of 3-(2-fluoro-2-methyl-propionylamino)-N-[(R)-1-(4-fluoro-phenyl)-ethyl]-5-(2-isoquinolin-3-yl-3H-imidazol-4-yl)-benzamide dihydrochloride

Scheme 5 illustrates the synthetic route to the intermediate 3-acetyl-5-nitro-benzoic acid [15]. 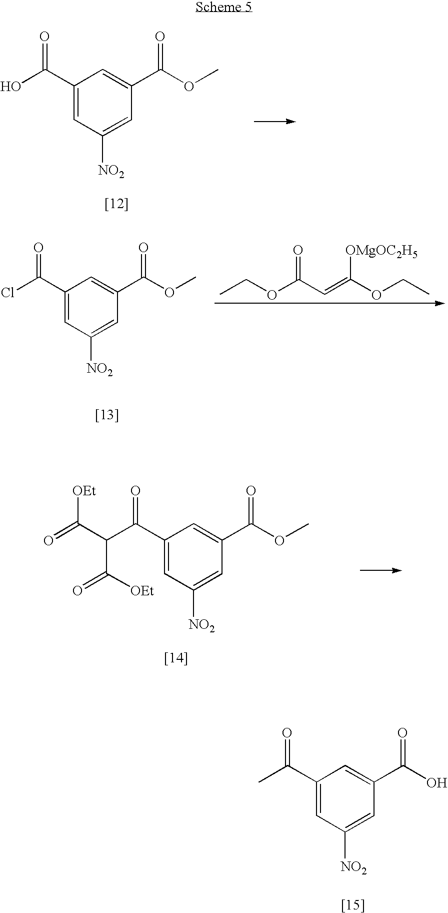
Intermediate 13

To 5-nitro-isophthalic acid monomethyl ester (1005.5 g, 4.466 mol) was added 6 L of anhydrous dichloroethane and 10 ml DMF. The mixture was warmed to 50-55° C. then SOCl₂ (425 ml, 5.8 mol) was added dropwise. The mixture was heated overnight at 70° C. The mixture was allowed to cool to rt, and the solvent was evaporated in vacuo. The residue was treated with 300 mL of toluene and concentrated. This procedure repeated twice further to afford the acid chloride Intermediate 13 (3-chlorocarbonyl-5-nitro-benzoic acid methyl ester) as a white solid.

Intermediate 14;

Mg(OEt)₂ (313 g, 2.73 mol) in THF (2 L) was treated with diethyl malonate (454.7 g, 2.84 mol) dropwise at rt. The mixture was refluxed overnight, then cooled to rt. The acid chloride, Intermediate 13 (634 g, 2.6 mol) was dissolved in THF (2 L) and added slowly to the mixture at rt. The resulting mixture was refluxed overnight. The mixture was cooled to rt, then slowly added to 2-2.5 L of rapidly stirring, chilled 1 N HCl. After the addition was complete, the product was extracted 2 times with ethyl acetate. The ethyl acetate layers were combined and concentrated under reduced pressure to produce an orange oil.

Intermediate 15

To Intermediate 14, 2-(3-methoxycarbonyl-5-nitro-benzoyl)-malonic acid diethyl ester (1.16 kg) was added a solution of H₂O/AcOH (1.46 L/2.2 L), then H₂SO₄(350 ml), in turn, and the mixture was refluxed overnight. The mixture was cooled to rt, then 10 L of H₂O was added and the product was extracted with ethyl acetate.

The ethyl acetate extract was washed with H₂O and then dried over Na₂SO₄. The ethyl acetate solution was concentrated in vacuo, and the residue was treated with 300 mL of toluene and concentrated; this procedure repeated twice further. The crude keto acid was recrystallized from 1:2 isopropanol-hexane. Refrigeration of the mixture followed by collection of the solid afforded 199 g of the product, Intermediate 15 (3-acetyl-5-nitro-benzoic acid).

Scheme 6 illustrates the synthetic route to the intermediate, N-[(R)-1-(4-fluoro-phenyl)-ethyl]-3-(2-isoquinolin-3-yl-3H-imidazol-4-yl)-5-nitro-benzamide dihydrochloride. 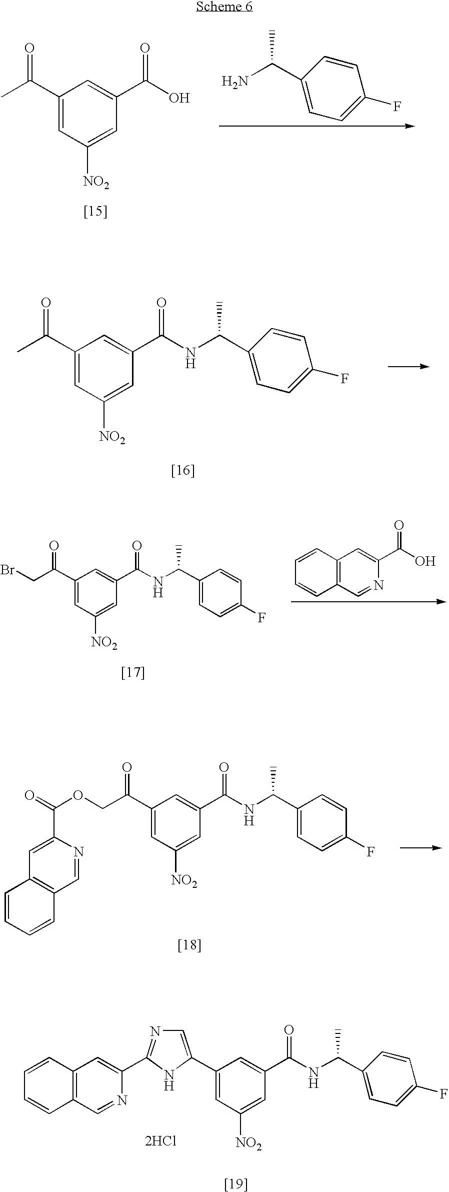
Intermediate 16

To a stirred mixture of Intermediate 15, 3-acetyl-5-nitro-benzoic acid (101.6 g, 0.486 mole) and HBTU (212.4 g, 0.56 mol) in DMF (1.5 L) at 0° C. was added DIEA (112 ml, 0.64 mole). The mixture was stirred at 0° C. for 30 min, followed by the addition of (R)-4-fluorophenethyl amine (69.5 g, 0.5 mol). The reaction mixture was stirred for 8 h at 0°0 C. and diluted with ice water and filtered. The collected solid was washed with water. The solid was dissolved in EtOAc and was washed with saturated NaHCO₃ solution and water. The organic layer was dried over Na₂SO₄ and concentrated in vacuo to give 155 g of Intermediate 16, 3-acetyl-N-[(R)-1-(4-fluoro-phenyl)-ethyl]-5-nitro-benzamide.

¹H NMR (CDCl₃): δ 8.82(2H), 8.68(1H), 7.36(2H), 7.16(1H), 7.00(2H), 5.30 (1H), 2.69(3H), 1.61(3H) ppm. LC/MS: m/z 373 (M+2)⁺

Intermediate 17

A solution of Intermediate 16, 3-acetyl-N-[(R)-1-(4-fluoro-phenyl)-ethyl]-5-nitro-benzamide (118 g, 0.36 mol) in 1,4-dioxane (500 ml) and DCM (100 ml) was treated with bromine (19.3 ml, 0.375 mol) dropwise over 35 min. The mixture was stirred at 10° C. for 2 hr. The solvent was removed in vacuo to afford 3-(2-bromo-acetyl)-N-[(R)-1-(4-fluoro-phenyl)-ethyl]-5-nitro-benzamide (412 g), which was used without further purification.

¹H NMR (CDCl₃): δ 8.83(2H), 8.71(1H), 7.34(2H), 7.03(3H), 5.30 (1H), 4.48(2H), 1.63(3H) ppm.

Intermediate 18

A mixture of isoquinoline-3-carboxylic acid monohydrate (63 g, 0.364 mol) and Na₂SO₄ (10 g) in dry DMF (200 mL) was treated with DIEA (76 mL, 0.44 mol). The reaction mixture was stirred at rt for 20 min and cooled to 0° C. followed by drop wise addition of Intermediate 17 (147 g, 0.36 mol) in DMF (200 ml). The mixture was stirred for 2 h at 0° C. and then diluted with ice water and filtered. The collected solid was washed with water then dissolved in EtOAc. The organic phase was washed with saturated aqueous NaHCO₃, then water, dried over Na₂SO₄ and concentrated. Hexane trituration afforded Intermediate 18 (155 g) (Isoquinoline-3-carboxylic acid 2-{3-[(R)-1-(4-fluoro-phenyl)-ethylcarbamoyl]-5-nitro-phenyl}-2-oxo-ethyl ester) as a white solid.

¹H NMR (CDCl₃): δ 9.34(1H), 8.68(1H), 8.08(2H), 8.01(1H), 7.80(2H), 7.75 (2H), 7.36(2H), 7.04(2H), 6.72(1H), 5.70(2H), 5.32(1H), 1.61(3H) ppm. LC/MS: m/z 503 (M+2)⁺

Intermediate 19

A solution of Intermediate 18, isoquinoline-3-carboxylic acid 2-{3-[(R)-1-(4-fluoro-phenyl)-ethylcarbamoyl]-5-nitro-phenyl}-2-oxo-ethyl ester (55 g, 0.11 mol) in AcOH (350 mL) and DMF (100 ml) was treated with ammonium acetate (127 g, 1.65 mol). The reaction mixture was stirred at 135-150° C. for 2 h. The mixture was cooled down to rt and treated with ice water. The resulting solid was filtered and washed with water. The solid was dissolved in EtOAc (350 ml) and washed with sat NaHCO₃ solution, then water was added. The mixture was shaken vigorously followed by the addition of 4N HCl (250 ml) and the mixture was shaken vigorously until the solid product began to precipitate. The solid was filtered, washed with water, and was dried under vacuum at 50° C. to afford 40 g of Intermediate 19 (N-[(R)-1-(4-Fluoro-phenyl)-ethyl]-3-(2-isoquinolin-3-yl-3H-imidazol-4-yl)-5-nitro-benzamide dihydrochloride).

¹H NMR (CD₃OD): δ 9.45(1H), 8.93(1H), 8.80(1H), 8.74(2H), 8.32(1H), 8.21(1H), 8.10(1H), 7.92 (1H), 7.85(1H), 7.48(2H), 7.08(2H), 5.28(1H), 1.64(3H) ppm. LC/MS: m/z 483 (M+2)⁺

Scheme 7 illustrates the synthetic route to N-[(1R)-1-(4-fluorophenyl)ethyl]-3-(2-isoquinoline-3-yl-1H-imidazol-4-yl)-5-(2-fluoroisobutyrylamino)-benzamide dihydrochloride. 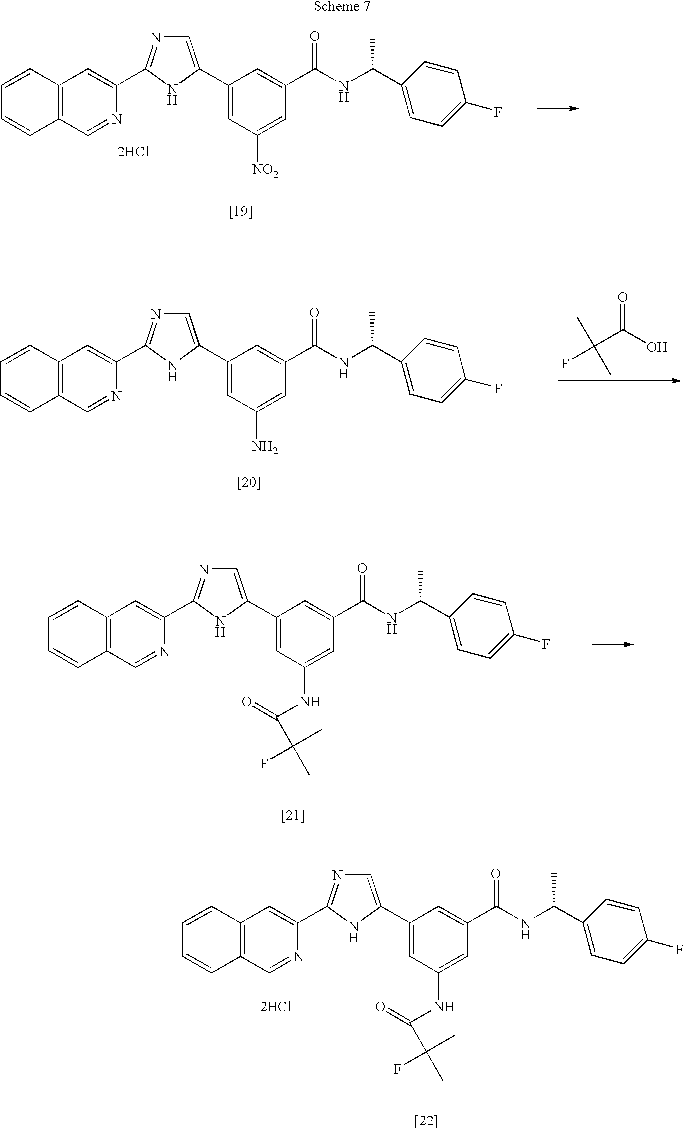
Intermediate 20

A solution of Intermediate 19, N-[(R)-1-(4-fluoro-phenyl)-ethyl]-3-(2-isoquinolin-3-yl-3H-imidazol-4-yl)-5-nitro-benzamide dihydrochloride (56 g, 0.1 mol) in MeOH-THF (200 mL-50 ml) under N₂ was treated with ammonium formate (38 g, 10.61 mol) and 10% palladium on carbon (8.5 g). The mixture was heated at 60° C. for 5 min, then the mixture was stirred at rt for 1 hr. The mixture was passed through a pad of filter aid (220 g) then concentrated and poured over ice-water. The solid was filtered, washed with water and dried under high vacuum at 40° C. to give 42.6 g of the desired product, 3-Amino-N-[(R)-1-(4-fluoro-phenyl)-ethyl]-5-(2-isoquinolin-3-yl-3H-imidazol-4-yl)-benzamide.

¹H NMR (CD₃OD): δ 9.25(1H), 8.69(1H), 8.42(1H), 8.03(1H), 7.93(1H), 7.75(2H), 7.60(3H), 7.43 (2H), 7.32(1H), 7.07(2H), 5.23(1H), 1.57(3H) ppm. LC/MS: m/z 453 (M+2)⁺

Intermediate 21

A stirred mixture of Intermediate 20, 3-amino-N-[(R)-1-(4-fluoro-phenyl)-ethyl]-5-(2-isoquinolin-3-yl-3H-imidazol-4-yl)-benzamide (42.6 g, 94.46 mmol) and HBTU (46.7 g, 122.8 mmol) in DMF (200 mL) at 0° C. was treated with DIEA (26 ml, 147.4 mmol). The mixture was stirred at 0° C. for 30 min, followed by the addition of 2-fluoroisobutyric acid (12 g, 113.35 mmol). The reaction mixture was stirred at 0° C. for 6 h and diluted with ice water, filtered and washed with water. The resulting solid was collected, dissolved in EtOAc, and washed with saturated NaHCO₃ and water. The organic layer was dried over Na₂SO₄ and concentrated in vacuo. The crude material was dissolved in EtOAc (60 ml) followed by the addition of hexane (100 ml). The solid was filtered and dried to give 39 g of the desired Intermediate 21 (Example 17).

Intermediate 21 (27.9 g, 51.76 mmol) was dissolved in 150 ml DCM and 20 ml MeOH and cooled to 0° C. HCl in dioxane (4 N, 28 ml) was added and the mixture was stirred at 0° C. for 1 h. The mixture was concentrated to about half of its original volume followed by the addition of hexane. The resulting solid was filtered, washed with DCM-hexane (5:1) and dried under vacuum at 50° C. to afford 30 g of N-[(1R)-1-(4-fluorophenyl)ethyl]-3-(2-isoquinoline-3-yl-1H-imidazol-4-yl)-5-(2-fluoroisobutyrylamino)-benzamide dihydrochloride.

¹H NMR (CD₃OD): δ 9.40(1H), 8.73(1H), 8.30(1H), 8.17(2H), 8.06(3H), 7.87(1H), 7.82(1H), 7.49 (2H), 7.07(2H), 5.25(1H), 1.68(6H), 1.61(3H) ppm. LC/MS: m/z 541(M+2)⁺ Biological Assay

The following assay methods may be used to identify compounds of Formula (I) that are effective in showing antiviral activity against vaccinia virus.

General Assay Procedures

Cytopathic effect was measured on the BSC40 african green monkey kidney cells using 100 μM concentrations of the compounds of Formula (I). In this assay, 96-well black Packard viewplates were seeded with BSC40 cells (2.25×10⁴ cells/well) in Minimum Essential Media supplemented with 5% FCS, 2 mM L-glutamine and 10 μg/mL gentamycin sulfate. When the cells became confluent (24 h) they were treated with 100 μM compound diluted in media. The cells were place in an incubator at 37° C. (5% CO₂) for 24 hours, and checked for toxicity via direct observation under the microscope and also with alamar blue which assesses cell viability and proliferation (healthy cells produce a visible color change from blue to red). The cells were scored on a scale of 0-3 where 0 corresponds to normal healthy cells, 1 corresponds to sick cells but not rounding up, 2 corresponds to cells that are rounding up, and 3 corresponds to cells that have rounded up and pulled off the plate. Compounds at concentrations that scored 1 or greater were diluted and the above assay was repeated to find the concentration at which the compound scored 0.

A vaccinia virus green fluorescent protein (vvGFP) assay was performed to test the ability of compounds of Formula (I) to inhibit viral growth as measured by a reduction in fluorescence from vaccinia virus expressing the green fluorescent protein. In this assay, 96-well black Packard viewplates were seeded with BSC40 cells in Minimum Essential Media supplemented with 5% FCS, 2 mM L-glutamine and 10 μg/mL gentamycin sulfate. When the cells became confluent, they were washed with PBS and then infected with vaccinia virus at a multiplicity of infection (moi) of 0.1 for 30 min in PBS. At 30 minutes, the cells were overlaid with 100 μl of infection media supplemented with 100 μM test compound. As controls infected cells are treated with rifampicin (blocks assembly of DNA and protein into mature virus particles), with no compound, or mock infected. Cells were placed in an incubator at 37° C. (5% CO₂) for 24 h. At 24 hours post infection (hpi), the plates were removed from the incubator, washed with PBS and fluorescence measure on a Wallac plate reader (excite at 485 nm and read at 535 nm). Wells that showed reduced fluorescence were checked visually under the microscope to verify a reduction in viral infection versus a loss of cells due to cytopathic effect from virus infection. Compounds that are found to inhibit viral replication were then checked for inhibitory effect at various concentrations to determine the EC₅₀ and the therapeutic index.

The compounds of Formula (I) listed in Table 1 have an EC₅₀ of less than or equal to about 100 μM. Various compounds such as Examples 1, 5, 6, 15, and 17 have an EC₅₀ of less than or equal to about 0.5 μM.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness of the subject being treated for a viral infection. Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

We claim:

1. A compound of Formula (I): 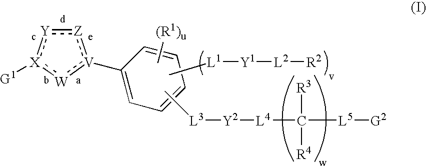
wherein
V is C, W is N—R¹¹, O, or S, X is C, Y is N, Z is C—R¹², when sides a, b, and d are single bonds, and sides c and e are double bonds;
V is C, W is N, X is C, Y is N—R¹¹, O, or S, Z is C—R¹², when sides a, c, and d are single bonds, and sides b and e are double bonds;
V is C, W is N, X is C, Y is C—R¹², Z is N—R¹¹, O, or S when sides b, e, and d are single bonds, and sides a and c are double bonds;
V is C, W is C—R¹², X is N, Y is C—R¹³, Z is N, when sides b, c, and e are single bonds, and sides a and d are double bonds;
V is N, W is C—R¹², X is C, Y is N, Z is C—R¹³, when sides a, c, and e are single bonds, and sides b and d are double bonds;
V is C, W is N—R¹¹, O, or S, X is C, Y is C—R¹², Z is N when sides a, b, and d are single bonds, and sides c and e are double bonds;
wherein
R¹¹ is R^{d};
R¹² and R¹³ are independently selected from R^{f};
G¹ is selected from the group consisting of: cycloalkyl, heterocyclyl, aryl, heteroaryl, fused arylcycloalkyl, fused cycloalkylaryl, fused cycloalkylheteroaryl, fused heterocyclylaryl, and fused heterocyclylheteroaryl group, wherein G¹ is optionally substituted with substituents independently selected from R⁵, wherein R⁵ is R^{b},
G² is selected from the group consisting of: cycloalkyl, heterocyclyl, aryl, heteroaryl, fused arylcycloalkyl, fused cycloalkylaryl, fused cycloalkylheteroaryl, fused heterocyclylaryl, and fused heterocyclylheteroaryl group, wherein G² is optionally substituted with substituents independently selected from R⁶, wherein R⁶ is R^{b},
R¹ is R^{b},
R¹² is R^{f},
R³ and R⁴ are independently selected from R^{f} and R^{g},
L¹, L², and L⁵ are independently selected from the group consisting of a direct bond, —C₁₋₁₀ alkylene, —C₂₋₁₀ alkenylene, and —C₂₋₁₀ alkynylene; wherein alkylene, alkenylene, and alkynylene are optionally substituted 1 to 4 times with R^{f},
L³ and L⁴ are independently selected from the group consisting of a direct bond, —C₁₋₁₀ alkylene, —C₂₋₁₀ alkenylene, —C₂₋₁₀ alkynylene, arylene, and heteroarylene; wherein alkylene, alkenylene, and alkynylene are optionally substituted 1 to 4 times with R^{f}, and arylene and heteroarylene are optionally substituted 1 to 4 times with R^{b},
Y¹ and Y² are independently selected from the group consisting of a direct bond, —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R⁷)—, —C(R^{f})═C(R^{g})—, —C≡C—, —N═N—, and —N(R¹⁶)—N(R¹⁷)—;
wherein
R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are optionally substituted 1 to 4 times with R^{f};
R^{b} is
a) -cycloalkyl,
b) -cyano,
c) —OR^{d},
d) —NO₂,
e) -halogen,
f) —S(O)ₘR^{d},
g) —SR^{d},
h) —S(O)₂OR^{d},
i) —S(O)ₘNR^{d}R^{e},
j) —NR^{d}R^{e},
k) —O(CR^{f}R^{g})ₙNR^{d}R^{e},
l) —C(O)R^{d},
m) —CO₂R^{d},
n) —CO₂(CR^{f}R^{g})ₙC(O)NR^{d}R^{e},
o) —OC(O)R^{d},
p) —C(O)NR^{d}R^{e},
q) —NR^{d}C(O)R^{e},
r) —OC(O)NR^{d}R^{e},
s) —NR^{d}C(O)OR^{e},
t) —NR^{d}C(O)NR^{d}R^{e},
u) —CF₃,
v) —OCF₃,
w) -haloalkyl,
x) -haloalkoxy,
y) —C₁₋₁₀ alkyl,
z) —C₂₋₁₀ alkenyl,
aa) —C₂₋₁₀ alkynyl,
bb) —C₁₋₁₀ alkylene-aryl,
cc) —C₁₋₁₀ alkylene-heteroaryl, or
dd) -heteroaryl,
wherein alkyl, alkenyl, alkynyl, aryl, heteroaryl, and cycloalkyl groups are optionally substituted 1-4 times with a group independently selected from R^{c};
R^{c} is
a) -halogen,
b) -amino,
c) -carboxy,
d) —C₁₋₄ alkyl,
e) —O—C₁₋₄ alkyl,
f) -cycloalkyl,
g) —O-cycloalkyl,
h) -aryl,
i) —C₁₋₄ alkylene-aryl,
j) -hydroxy,
k) —CF₃,
l) -haloalkyl,
m) -haloalkoxy,
n) —O-aryl,
o) -heteroaryl,
p) -heteroaryl-C₁₋₁₀ alkyl,
q) heterocyclyl,
r) —CO₂—C₁₋₁₀ alkyl, or
s) —CO₂—C₁₋₁₀ alkyl-aryl,
R^{d} and R^{e} are independently selected from hydrogen, C₁₋₁₀ alkyl, C₂₋₁₀ alkenyl, C₂₋₁₀ alkynyl, cycloalkyl, —C₁₋₁₀ alkylene-cycloalkyl, aryl, heterocyclyl, wherein alkyl, alkenyl, alkynyl, cycloalkyl, aryl, heterocyclyl groups are optionally substituted with one to four substituents independently selected from R^{c}; or R^{d}and R^{e} together with the atoms to which they are attached form a heterocyclic ring of 5 to 7 members containing 0-2 additional heteroatoms independently selected from oxygen, sulfur and nitrogen and optionally substituted with 1-3 times with R^{c},
R^{f} and R^{g} are independently selected from hydrogen, C₁₋₁₀ alkyl, cycloalkyl, —C₁₋₁₀ alkylene-cycloalkyl, and aryl, wherein alkyl, cycloalkyl, and aryl groups are optionally substituted with one to four substituents independently selected from R^{c}; or R^{f} and R^{g} together with the carbon to which they are attached form a ring of 5 to 7 members containing 0-2 heteroatoms independently selected from oxygen, sulfur and nitrogen optionally substituted with 1-3 times with R^{c};
m is an integer from 1 to 2,
n is an integer from 1 to 10,
u is an integer from 0 to 2,
v is an integer from 0 to 2,
w is an integer from 0 to 1,
or pharmaceutically acceptable salt, solvate, or prodrug thereof.

2. The compound of Formula (I) in claim 1 or a pharmaceutically acceptable salt thereof, wherein G¹ is isoquinoline-3-yl, quinoline-2-yl, quinoline-3-yl, pyridine-2-yl, pyridine-3-yl, phenyl, or naphthyl-2-yl, wherein G¹ may be substituted or unsubstituted.

3. The compound of Formula (I) in claim 1 or a pharmaceutically acceptable salt thereof, wherein G¹ is isoquinoline-3-yl, pyridine-2-yl, or pyridine-3-yl, wherein G¹ is unsubstituted.

4. The compound of Formula (I) in claim 1 or a pharmaceutically acceptable salt thereof, wherein G² is phenyl, pyridine, pyrimidine, pyridazine, or pyrazine,
wherein G² is optionally substituted 1 to 4 times with R⁶, wherein R⁶ is R^{b}.

5. The compound of Formula (I) in claim 1 or a pharmaceutically acceptable salt thereof, wherein V is C, W is N—R¹¹, O, or S, X is C, Y is N, Z is C—R¹², sides a, b, and d are single bonds, and sides c and e are double bonds.

6. The compound of Formula (I) in claim 1 or a pharmaceutically acceptable salt thereof, wherein V is C, W is N—R¹¹, X is C, Y is N, Z is C—R¹², sides a, b, and d are single bonds, and sides c and e are double bonds.

7. The compound of Formula (I) in claim 6 or a pharmaceutically acceptable salt thereof, wherein R¹¹ and R¹² are hydrogen.

8. The compound of Formula (I) in claim 1 or a pharmaceutically acceptable salt thereof, wherein u is 0, v is 1, L¹ is a direct bond, L² is a direct bond, Y¹ is —N(R¹⁶)C(O)—, and R¹² is R^{f}.

9. The compound of Formula (I) in claim 1 or a pharmaceutically acceptable salt thereof, wherein u is 0, v is 1, L¹ is a direct bond, L² is a direct bond,
R¹² is hydrogen, C₁₋₁₀ alkyl, cycloalkyl, phenyl, or —C₁₋₁₀ alkylene-phenyl, wherein alkyl, cycloalkyl, and phenyl are substituted or unsubstituted, and
Y¹ is —N(R¹⁶)C(O)—, wherein R¹⁶ is hydrogen, C₁₋₁₀ alkyl, cycloalkyl, or phenyl, wherein alkyl, cycloalkyl, and phenyl are substituted or unsubstituted.

10. The compound of Formula (I) in claim 1 or a pharmaceutically acceptable salt thereof, wherein u is 0, v is 1, L¹ is a direct bond, L² is a direct bond,
Y¹ is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶)—, —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, and —C(R^{f})═C(R^{g})—;
wherein
R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted; and
R¹² is C₁₋₁₀ alkyl, cycloalkyl, phenyl, or —C₁₋₁₀ alkylene-phenyl, wherein alkyl, cycloalkyl, and phenyl are substituted or unsubstituted.

11. The compound of Formula (I) in claim 1 or a pharmaceutically acceptable salt thereof, wherein u is 0, v is 1, L¹ is a direct bond, L² is a direct bond,
Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted; and
R¹² is C₁₋₁₀ alkyl, -cycloalkyl, -phenyl, or —C₁₋₁₀ alkylene-phenyl, wherein alkyl, cycloalkyl, and phenyl are substituted or unsubstituted.

12. The compound of Formula (I) in claim 1 or a pharmaceutically acceptable salt thereof, wherein u is 0, v is 1, L¹ is a direct bond, L² is a direct bond,
Y¹ is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted; and
R¹² is methyl, ethyl, propyl, isopropyl, tert-butyl, isobutyl, 1-ethyl-propyl, and (1-halo-1-methyl)-ethyl.

13. The compound of Formula (I) in claim 1 or a pharmaceutically acceptable salt thereof, wherein L³, L⁴, and L⁵ are direct bonds, and w is 1.

14. The compound of Formula (I) in claim 1 or a pharmaceutically acceptable salt thereof, wherein L³, L⁴, and L⁵ are direct bonds, w is 1, and
Y² is selected from the group consisting of a —O—, —N(R¹⁶)—, —C(O)—, —C(O)N(R¹⁶)—, —N(R¹⁶)C(O)—, —N(R¹⁶)C(O)N(R¹⁷)—, —N(R¹⁶)C(O)O—, —OC(O)N(R¹⁶), —N(R¹⁶)SO₂—, —SO₂N(R¹⁶)—, —C(O)—O—, —O—C(O)—, —S—, —S(O)—, —S(O)₂—, —N(R¹⁶)SO₂N(R¹⁷)—, and —C(R^{f})═C(R^{g})—;
wherein
R¹⁶ and R¹⁷ are independently selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.

15. The compound of Formula (I) in claim 1 or a pharmaceutically acceptable salt thereof, wherein L³, L⁴, and L⁵ are direct bonds, w is 1, and
Y² is selected from the group consisting of a —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -aryl, -cycloalkyl, and —C₁₋₁₀ alkylene-aryl, wherein alkyl, cycloalkyl, and aryl are substituted or unsubstituted.

16. The compound of Formula (I) in claim 1 or a pharmaceutically acceptable salt thereof, wherein L³, L⁴, and L⁵ are direct bonds, w is 1,
Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted;
R³ is H,
R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
G² is phenyl substituted from 1 to 4 times with R⁶.

17. The compound of Formula (I) in claim 1 or a pharmaceutically acceptable salt thereof, wherein L³, L⁴, and L⁵ are direct bonds, w is 1,
Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
R³ is H,
R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
G² is phenyl substituted from 1 to 4 times with R⁶, wherein G² is substituted with at least one halogen.

18. The compound of Formula (I) in claim 1 or a pharmaceutically acceptable salt thereof, wherein L³, L⁴, and L⁵ are direct bonds, w is 1,
Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are substituted or unsubstituted;
R³ is H,
R⁴ is —C₁₋₁₀ alkyl, -cycloalkyl, —C₁₋₁₀ alkylene-cycloalkyl, and -aryl, wherein alkyl, cycloalkyl, and aryl groups are substituted or unsubstituted, and
G² is para-halophenyl.

19. The compound of Formula (I) in claim 1 or a pharmaceutically acceptable salt thereof, wherein the group -L³-Y²-L⁴-(C(R³)(R⁴))-L⁵-G² is taken together to form the group 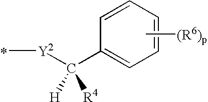
wherein
Y² is selected from the group consisting of —C(O)N(R¹⁶)—, —N(R¹⁶)C(O)—, —N(R¹⁶)SO₂—, —SO₂N(R¹⁶)—, —C(O)—O—, —O—C(O)—, and —C(R^{f})═C(R^{g})—;
wherein
R¹⁶ is selected from the group consisting of: -hydrogen, —C₁₋₁₀ alkyl, -cycloalkyl, -aryl, and —C₁₋₁₀ alkylene-aryl, wherein alkyl, cycloalkyl, and aryl are optionally substituted with R^{f};
R⁴ is R^{b},
R⁶ is R^{b}, and
p is an integer from 0 to 4.

20. The compound of Formula (I) in claim 27 or a pharmaceutically acceptable salt thereof, wherein Y² is —C(O)NH—, R⁴ is —C₁₋₄ alkyl, p is 1, and R⁶ is halo.

21. The compound of Formula (I) in claim 1 or a pharmaceutically acceptable salt thereof, wherein the group -L³-Y²-L⁴-(C(R³)(R⁴))-L⁵-G² is taken together to form the group 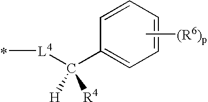
wherein
L⁴ is imidazole, oxazole, or thiazole,
R⁴ is R^{b},
R⁶ is R^{b}, and
p is an integer from 0 to 4.

22. The compound of Formula (I) in claim 29 or a pharmaceutically acceptable salt thereof, wherein Y² is imidazole, R⁴ is —C₁₋₄ alkyl, p is 1, and R⁶ is halo.

23. The compound of Formula (I) in claim 1 or a pharmaceutically acceptable salt thereof, wherein the compound of Formula (I) has the formula (Ia) 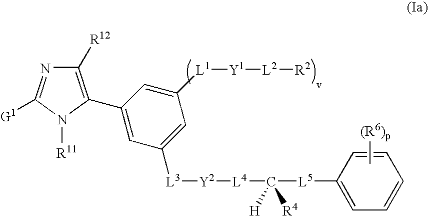
wherein
G¹, R¹², R⁴, R⁵, R¹¹, R¹², L¹, L², L³, L⁴, L⁵, Y¹, Y², and v are as defined in claim 1, and p is an integer from 0 to 4.

24. The compound of Formula (Ia) in claim 23 or a pharmaceutically acceptable salt thereof, G¹ is isoquinoline-3-yl, quinoline-2-yl, quinoline-3-yl, pyridine-2-yl, pyridine-3-yl, phenyl, or naphthyl-2-yl, wherein G¹ may be substituted or unsubstituted.

25. The compound of Formula (Ia) in claim 23 or a pharmaceutically acceptable salt thereof, wherein G¹ is isoquinoline-3-yl, pyridine-2-yl, or pyridine-3-yl, wherein G¹ is unsubstituted.

26. The compound of Formula (Ia) in claim 23 or a pharmaceutically acceptable salt thereof, wherein R¹¹ and R¹² are independently selected from the group consisting of hydrogen, C₁₋₁₀ alkyl, cycloalkyl, phenyl, and —C₁₋₁₀ alkylene-phenyl.

27. The compound of Formula (Ia) in claim 23 or a pharmaceutically acceptable salt thereof, wherein R¹¹ and R¹² are hydrogen.

28. A pharmaceutical composition comprising a compound of Formula (I) in claim 1 or a pharmaceutically acceptable salt thereof, and a pharmaceutically acceptable carrier, excipient, diluent, or mixture thereof.

29. The pharmaceutical composition of claim 28, further comprising a therapeutically effective amount of the compound of Formula (I) or a pharmaceutically acceptable salt thereof.

30. The pharmaceutical composition of claim 29, further comprising one or more additional therapeutic agents.

31. The pharmaceutical composition of claim 30, wherein the additional therapeutic agent is an antiviral agent selected from the group consisting of: AZT, abacavir, ddC, ddI, d4T, 3TC, ZDV, tenofovir, nevirapine, pentafuside, amprenavir, fosamprenavir, indinavir, nelfinavir, ritonavir, saquanivir, lamivudine, foscarnet, acyclovir, cidofovir, ganciclovir, valaciclovir, amantadine, rimantadine, zanamivir, and oseltamivir.

32. The pharmaceutical composition of claim 28 in the form of an oral, rectal, nasal, topical (including ocular, buccal and sublingual), vaginal or parenteral (including subcutaneous, intramuscular, intravenous, intradermal, intrathecal and epidural) dosage.

33. The pharmaceutical composition of claim 28, wherein said compound of Formula (I) is a dose of less than 1,000 mg/kg of body weight per day.

34. A method comprising administering to a subject the compound of Formula (I) in claim 1, or a pharmaceutically acceptable salt thereof.

35. The method of claim 34, wherein the compound of Formula (I) or a pharmaceutically acceptable salt thereof is an amount sufficient to reduce a viral load in a subject.

36. The method of claim 34, wherein said compound of Formula (I) or a pharmaceutically acceptable salt thereof is administered as a dose of less than 1,000 mg/kg of body weight of the subject per day.

37. A method of treating a viral condition comprising administering to a subject in need thereof a therapeutically effective amount of a compound of Formula (I) in claim 1 or a pharmaceutically acceptable salt thereof or a pharmaceutical composition comprising a compound of Formula (I) or a pharmaceutically acceptable salt thereof.

38. The method of treating a viral condition of claim 37, wherein the compound of Formula (I) or a pharmaceutically acceptable salt thereof or the pharmaceutical composition comprising a compound of Formula (I) or a pharmaceutically acceptable salt thereof is administered to a subject prophylactically, or prior to the onset of or diagnosis of a viral infection.

39. The method of claim 37, wherein the viral condition is associated with a virus selected from the group consisting of:
Adenoviridae including adenovirus, Hepadnaviridae including hepatitis B virus (HBV), Herpesviridae including herpes simplex virus type 1 (HSV-1), type 2 (HSV-2), thymidine kinase-deficient (TK⁻) HSV-1, varicella-zoster virus (TK⁺ and TK⁻ VZV), cytomegalovirus (CMV), human herpesvirus type 6 (HHV-6), and feline herpesvirus, Poxviridae including vaccinia virus, Papillomaviridae including human papilloma virus, and Polyomaviridae including polyoma virus.

40. The method of claim 37, wherein the viral condition is associated with a virus selected from the group consisting of:
Retroviridae including human immunodeficiency virus type 1 (HIV-1) and type 2 (HIV-2), simian immunodeficiency virus (SIV), and moloney murine sarcoma virus, Coronaviridae including feline (FIPV) corona virus, human (SARS) CoV, and mouse hepatitis virus, Flaviviridae including flavivirus (yellow fever virus (YFV), dengue-type 2 virus, and modoc virus (murine flavivirus)), hepacivirus (hepatitis A, B, or C), and pestivirus (bovine viral diarrhea virus (BVDV)), Picornaviridae including coxsackie B virus, polio virus, and rhinovirus, Alphaviridae including sindbis virus, Arenaviridae including arenaviruses (Tacaribe), Bunyaviridae including punta toro, Orthomyxoviridae including influenza A, B, and C virus, Paramyxoviridae including respiratory syncytial virus (RSV) and parainfluenza-3 virus, and Reoviridae including reo-1 virus.

41. The method of claim 37, wherein the viral condition is associated with a virus selected from the group consisting of: Poxviridae including vaccinia virus.

42. The method of claim 37, wherein the compound of Formula (I) or a pharmaceutically acceptable salt thereof is administered as a pharmaceutical composition and the pharmaceutical compositions is administered orally, rectally, nasally, topically (including ocular, buccal and sublingual), vaginally or parenterally (including subcutaneous, intramuscular, intravenous, intradermal, intrathecal and epidural).

43. The method of claim 42, wherein the pharmaceutical composition is administered orally.

44. The method of claim 37, wherein the compound of Formula (I) or a pharmaceutically acceptable salt thereof is administered as a dose of less than 1,000 mg/kg of body weight of the subject per day.

45. The method of claim 37, wherein the compound of Formula (I) or a pharmaceutically acceptable salt thereof is administered in conjunction with one or more other therapeutic agents used to treat conditions associated with a viral infection in a subject.

46. The method of claim 45, wherein the additional therapeutic agent is selected from the group consisting of: AZT, abacavir, ddC, ddI, d4T, 3TC, ZDV, tenofovir, nevirapine, pentafuside, amprenavir, fosamprenavir, indinavir, nelfinavir, ritonavir, saquanivir, lamivudine, foscarnet, acyclovir, cidofovir, ganciclovir, valaciclovir, amantadine, rimantadine, zanamivir, and oseltamivir.

47. A method for inhibiting propagation of a virus comprising administering to a subject in need thereof a compound of Formula (I) in claim 1 or a pharmaceutically acceptable salt thereof.

48. The method of claim 47, wherein the virus is selected from the group consisting of Poxviridae including vaccinia virus.
